(19) United States
(12) Patent Application Publication
(10) Pub. No.: US 2016/0151059 A1
(43) Pub. Date: Jun. 2, 2016

(54) SYSTEM AND METHOD FOR IMAGE-GUIDED ARTHROSCOPY

(71) Applicant: Reprise Technologies, LLC, Menlo Park, CA (US)

(72) Inventors: Frank Litvack, Los Angeles, CA (US); Robert C. Klapper, Los Angeles, CA (US); John F. Shanley, Emerald Hills, CA (US)

(73) Assignee: Reprise Technologies, LLC, Menlo Park, CA (US)

(21) Appl. No.: 15/014,522

(22) Filed: Feb. 3, 2016

Related U.S. Application Data
(63) Continuation of application No. 14/675,417, filed on Mar. 31, 2015, now abandoned.
Application No. 14/675,417 is a continuation of application No. 13/859,630, filed on Apr. 9, 2013, now abandoned.
Application No. 13/859,630 is a continuation of application No. 13/030,109, filed on Feb. 17, 2011, now abandoned.
(60) Provisional application No. 61/305,519, filed on Feb. 17, 2010.
(60) Provisional application No. 61/311,117, filed on Mar. 5, 2010.
(60) Provisional application No. 61/315,795, filed on Mar. 19, 2010.
(60) Provisional application No. 61/377,670, filed on Aug. 27, 2010.

(51) Int. Cl. A61B 17/02; A61B 17/34

(52) U.S. Cl. CPC A61B 17/0218; A61B 17/3423; A61B 2017/0225; A61B 2017/3486

(57) ABSTRACT

Configurations are described for conducting minimally invasive medical diagnoses and interventions utilizing elongate instruments and assemblies comprising one or more imaging devices, and one or more remote retraction and distraction devices. Retraction and distraction devices, such as balloons, mechanical retraction members, and/or trocar screw geometries may be utilized to access, investigate, and intervene at the joint capsule, or inside of the joint capsule. Imaging devices, such as optical image capture devices, ultrasound transducers, and optical coherence tomography fibers, may be utilized to assist with navigation of the pertinent tools during diagnostic and interventional steps.

### RELATED APPLICATION DATA

This application is a continuation of U.S. patent application Ser. No. 14/675,417, filed on Mar. 31, 2015, which is a continuation of U.S. patent application Ser. No. 13/859,630, filed on Apr. 9, 2013, which is a continuation of U.S. patent application Ser. No. 13/030,109, filed on Feb. 17, 2011, which claims the benefit under 35 U.S.C. §119 to U.S. provisional patent application Ser. Nos. 61/305,519, filed Feb. 17, 2010, 61/311,117, filed Mar. 5, 2010, 61/315,795, filed Mar. 19, 2010, and 61/377,670, filed Aug. 27, 2010. The foregoing applications are hereby incorporated by reference into the present application in their entirety.

### FIELD OF THE INVENTION

The present invention relates to diagnostic and/or interventional arthroscopy, and particularly to the utilization of various imaging, retraction, and distraction technologies as coupled to elongate arthroscopy instruments for improved visualization, navigation, access, and guidance during clinical procedures.

### BACKGROUND

Hundreds of thousands of patients in the US and other countries undergo diagnostic and/or interventional arthroscopy every year. For example, arthroscopy of the knee joint, hip joint, and elbow joint is relatively common, and generally is associated with an intervention related to the soft tissue structures comprising or supporting such joint, such as the ligaments or articular surfaces. Arthroscopy typically involves an elongate instrument configured to capture images of adjacent tissue structures and other instruments in situ—typically using optical or digital camera technologies. One of the challenges with camera technologies is that they are not very well suited for imaging through blood, fascia, or other tissues which may not transmit light as well as saline, synovial fluid, or gases such as nitrogen, carbon dioxide, or air. It would be desirable to have arthroscopy tools that have on board imaging capabilities which are configured to see through and/or past such structures. For example, arthroscopy of the hip presents the clinician with various challenges, including the desire to have minimal port wounds and port sizes, as well as minimal tissue damaged by virtue of insertion, use, and retraction of the various elongate instruments which may be brought into the in situ surgical theater. Referring to FIG. 1A, some elements of the hip anatomy are depicted, including the femur (10), femoral head (8), acetabulum (6), articular cartilage (4), ilium (2), medial aspect of the joint capsule (24), and lateral aspect of the joint capsule (26). FIG. 1D depicts a diagrammatic representation of similar structures for illustration purposes. FIGS. 1B and 1C illustrate conventional arthroscopy tool assemblies (12, 13) which may be utilized in hip interventional or diagnostic procedures. Referring to FIG. 1C, each assembly typically comprises a handle (14, 15), a signal conduit (18, 19) configured to provide power and/or light and transmit images through electronic cables or fibers, a saline or fluid cycling conduit (16, 17), and an elongate distal portion (20, 21) configured to reach the pertinent anatomy while minimizing damage and maximizing freedom of surgical motion. Typically one or more of such instrument assemblies will have an imaging fiber bundle to transmit captured light signals to a proximal location where they may be digitized, for example by a digital imaging chip, and/or magnified with one or more lenses; alternatively, a digital image capture chip may be disposed distally, with leads coupling such chip to a proximal image processing system. Such image capture configurations typically will have a forward (i.e., at least somewhat parallel with the longitudinal axis of the elongate distal portion 20, 21) oriented field of view. Such configurations are utilized for hip arthroscopy, and arthroscopy of other relatively dense and challenging joints, such as the elbow, but as discussed above, the image capture devices are not capable of seeing past blood or tissue or making measurements associated with such tissues. It would be valuable to have arthroscopy tools which are configured to image not only through various tissues such as blood and fascia, but also at various positions and/or angles other than the conventional forward oriented field of view configuration. Further, it would be valuable to have tools to assist with distraction of the subject joint, and retraction of nearby tissues, during diagnostic and/or interventional procedures.

### BRIEF DESCRIPTION OF THE DRAWINGS

FIG. 1A illustrates aspects of hip joint anatomy.

FIGS. 1B-1C illustrate hip arthroscopy tools which may be utilized in hip interventions.

FIG. 1D illustrates a diagrammatic illustration of hip joint anatomy.

FIGS. 2A-2C illustrate aspects of a hip joint with labral impingement.

FIGS. 3A through 3Z-9 illustrate aspects of a hip arthroscopy intervention wherein a labrum is debrided with remote distraction, and wherein a femoral neck geometry is modified.

FIGS. 4A-4G illustrate aspects of a hip arthroscopy intervention wherein a labrum is debrided with remote distraction, and wherein a femoral neck geometry is modified, using a single port access configuration.

FIGS. 5A through 5Z-5B illustrate various aspect of configuration options for interventional tools and techniques pertinent to embodiments such as those described in reference to FIGS. 3A through 3Z-9 and 4A-4G.

FIGS. 6A-6C illustrates various aspects of arthroscopy methods in accordance with the present invention.

### SUMMARY OF THE INVENTION

One embodiment is directed to an apparatus for conducting an orthopaedic intervention, comprising an elongate trocar member having proximal and distal ends, a longitudinal axis, and defining a first working lumen therethrough; and a controllably expandable distal member coupled to the distal end of the elongate trocar member and configured to be expanded from a collapsed state, wherein the expandable distal member has an outer diameter similar to that of the trocar member, to an expanded state, wherein the expandable distal member has an outer diameter larger than that of the trocar member. The apparatus may further comprise an image capture device coupled to the distal end of the elongate trocar member and configured to have a field of view extending away from the distal end of the elongate trocar member and at least partially in alignment with the longitudinal axis of the elongate trocar member. The elongate trocar member may be substantially rigid. The elongate trocar member may have a structural modulus configured to maintain a shape substantially similar to an unloaded configuration of the trocar member when navigated through soft tissues, but to deform in sympathy to loads imparted by skeletal calcified tissues with which it comes into contact. The controllably expandable distal member may comprise an inflatable balloon member. The elongate trocar member may comprise an inflation lumen fluidly coupled to the inflatable balloon member. The inflatable balloon member expanded state may define a substantially rounded outer shape. The inflatable balloon member expanded state may comprise a frustoconical shape. The frustoconical shape may define a substantially empty working volume adjacent to the distal end of the elongate trocar member distal end. The apparatus may further comprise an image capture device coupled to the distal end of the elongate trocar member and configured to have a field of view extending away from the distal end of the elongate trocar member and at least partially in alignment with the longitudinal axis of the elongate trocar member, wherein the field of view of the image capture device captures a substantial portion of the working volume defined by the frustoconical shape. The expandable distal member may comprise a flexible cuff. The flexible cuff may be biased to self-expand to the expanded state when not restrained in the collapsed state. The flexible cuff expanded state may comprise a frustoconical shape. The frustoconical shape may define a substantially empty working volume adjacent to the distal end of the elongate trocar member distal end. The apparatus may further comprise an image capture device coupled to the distal end of the elongate trocar member and configured to have a field of view extending away from the distal end of the elongate trocar member and at least partially in alignment with the longitudinal axis of the elongate trocar member, wherein the field of view of the image capture device captures a substantial portion of the working volume defined by the frustoconical shape. The image capture device may comprise an ultrasound transducer. The image capture device may comprise an optical imaging device. The image capture device may comprise an optical coherence tomography imaging fiber. The elongate trocar member may further comprise a transparent fluid reservoir fluidly coupled to the working volume and configured to controllably flush the working volume with transparent fluid, thereby substantially clearing the volume of nontransparent fluids. The controllably expandable distal member may comprise one or more cutting elements configured to lacerate nearby soft tissue structures when in the expanded state and urged against said nearby soft tissue structures. At least one of the one or more cutting elements may comprise a mechanical feature that becomes prominently exposed when the controllably expandable distal member is in the expanded state.

### DETAILED DESCRIPTION

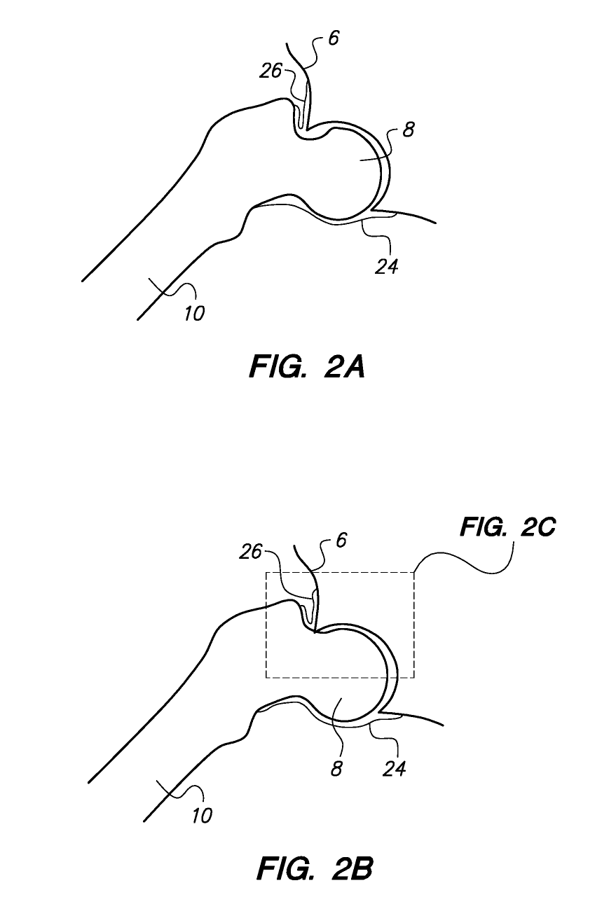

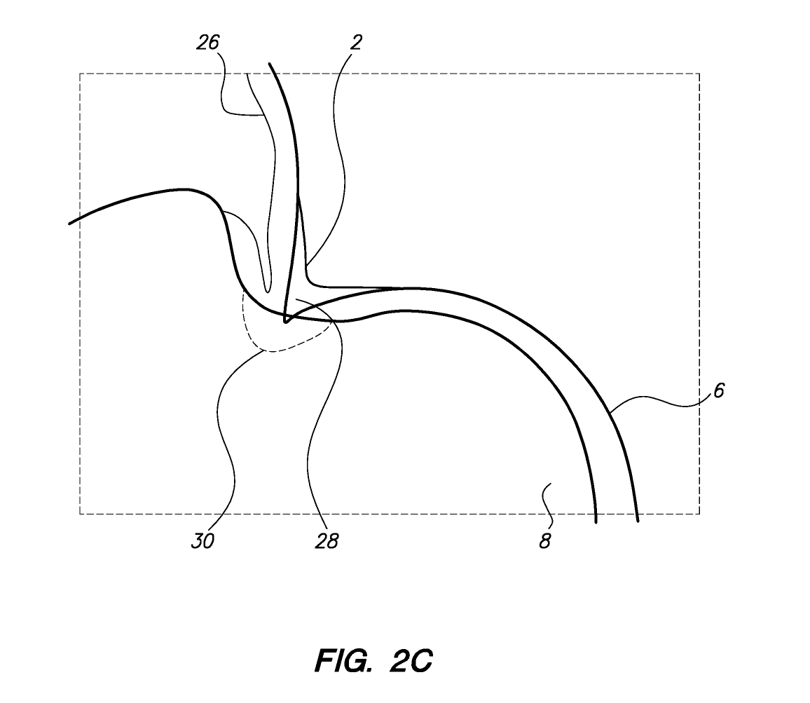

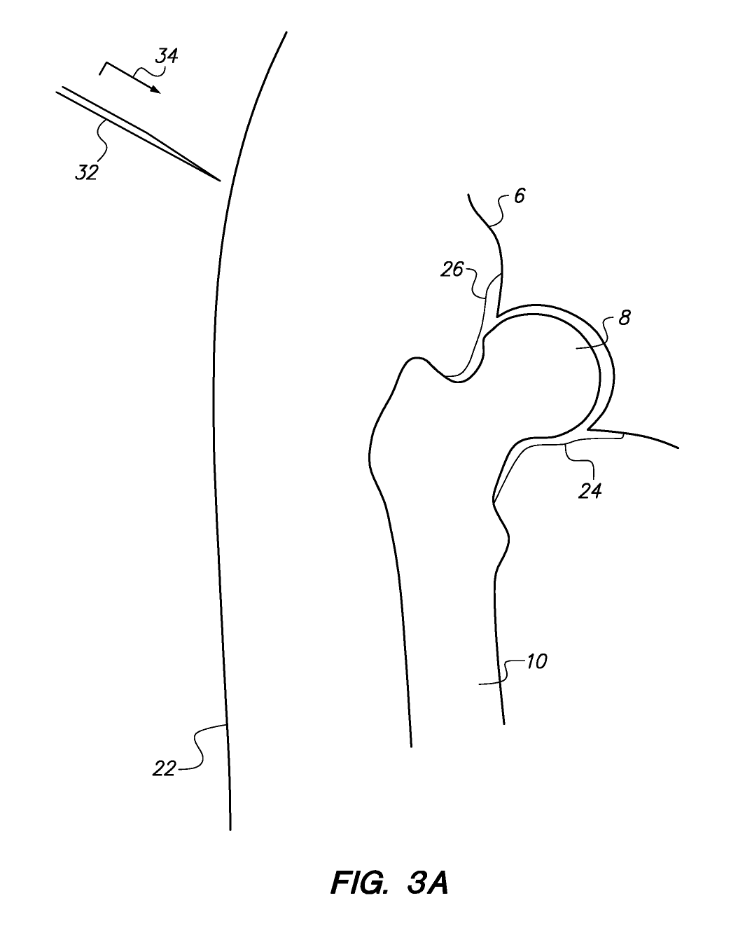

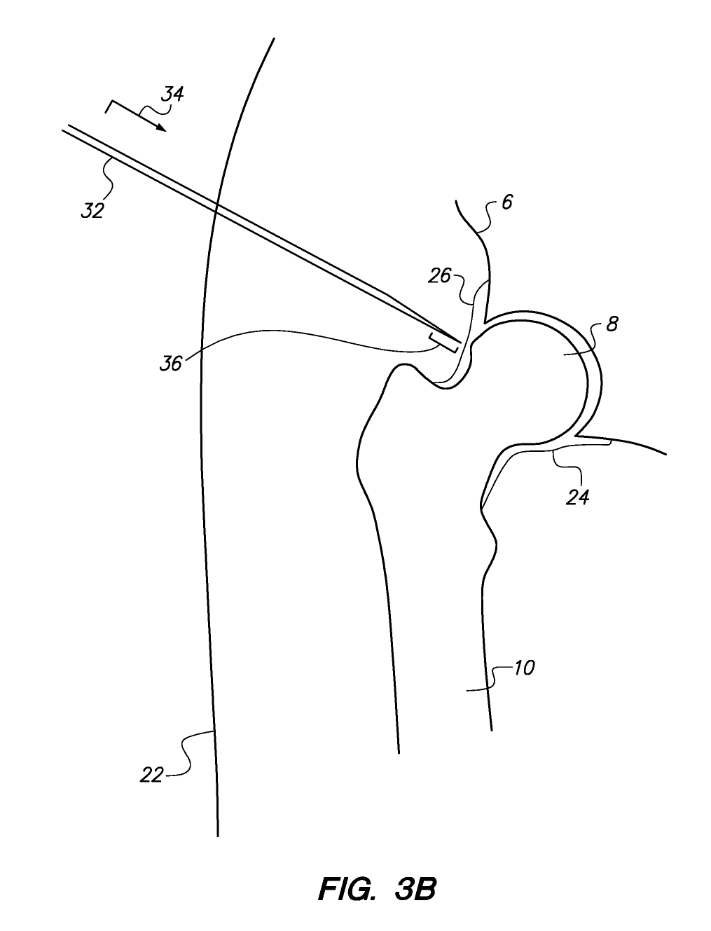

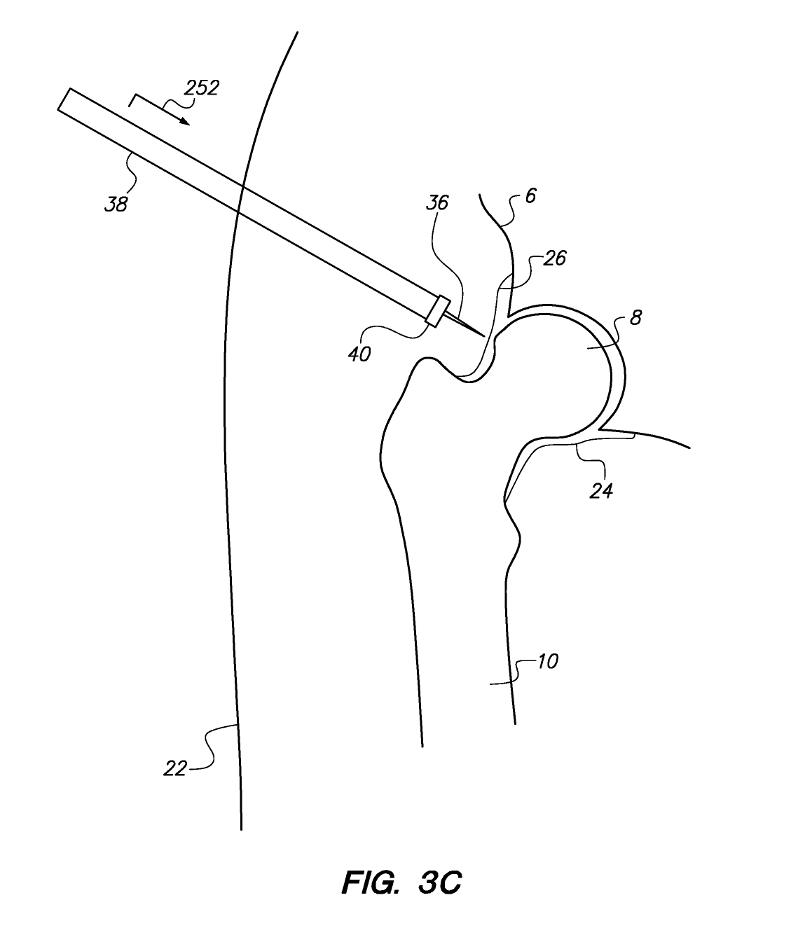

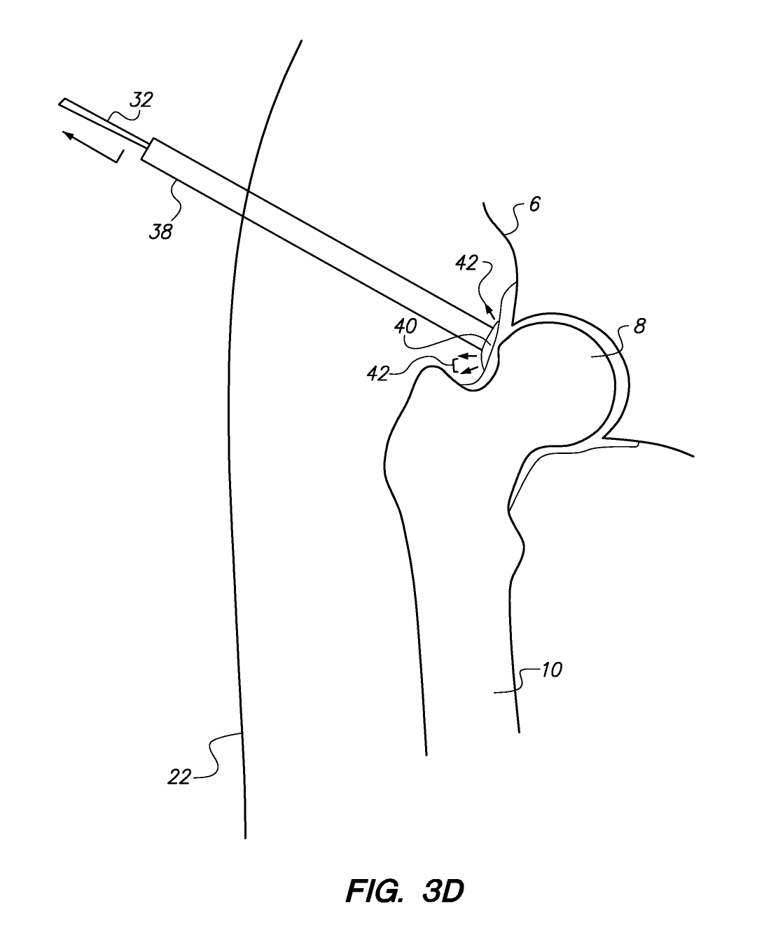

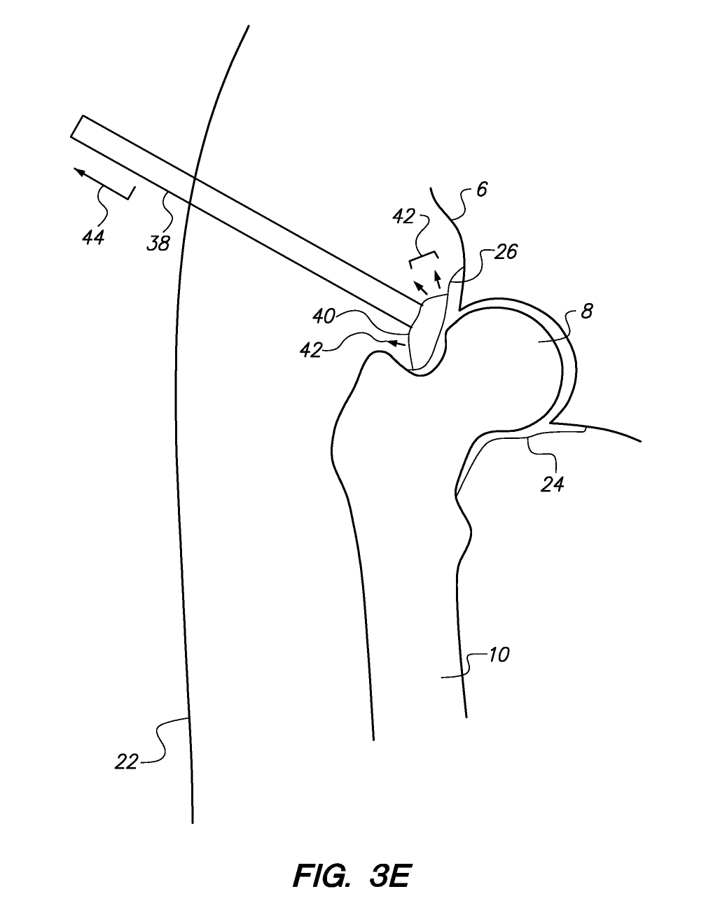

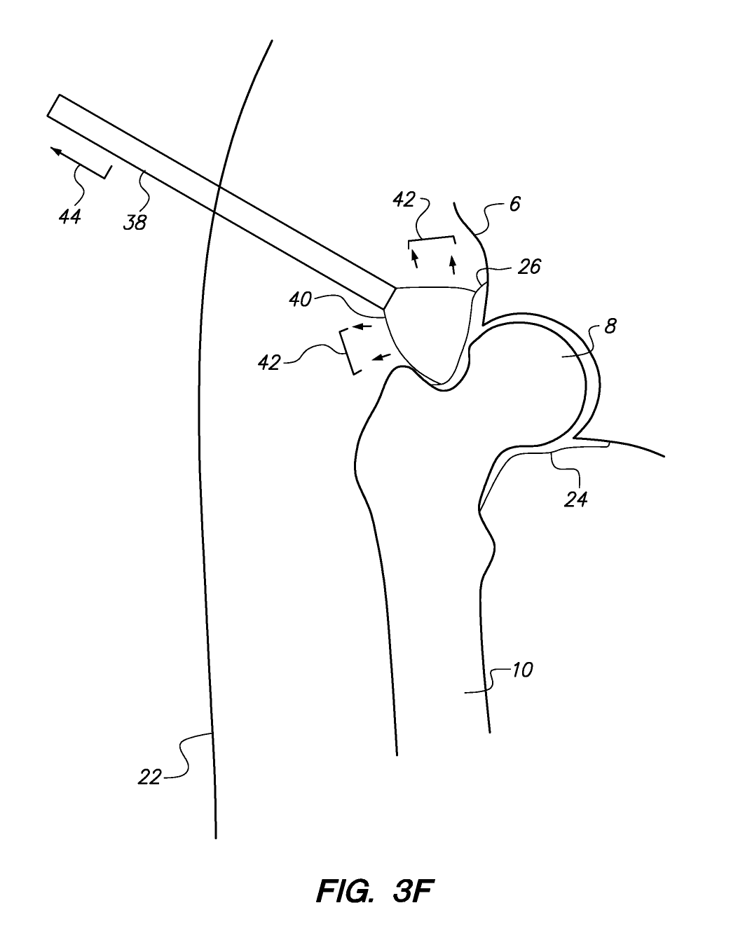

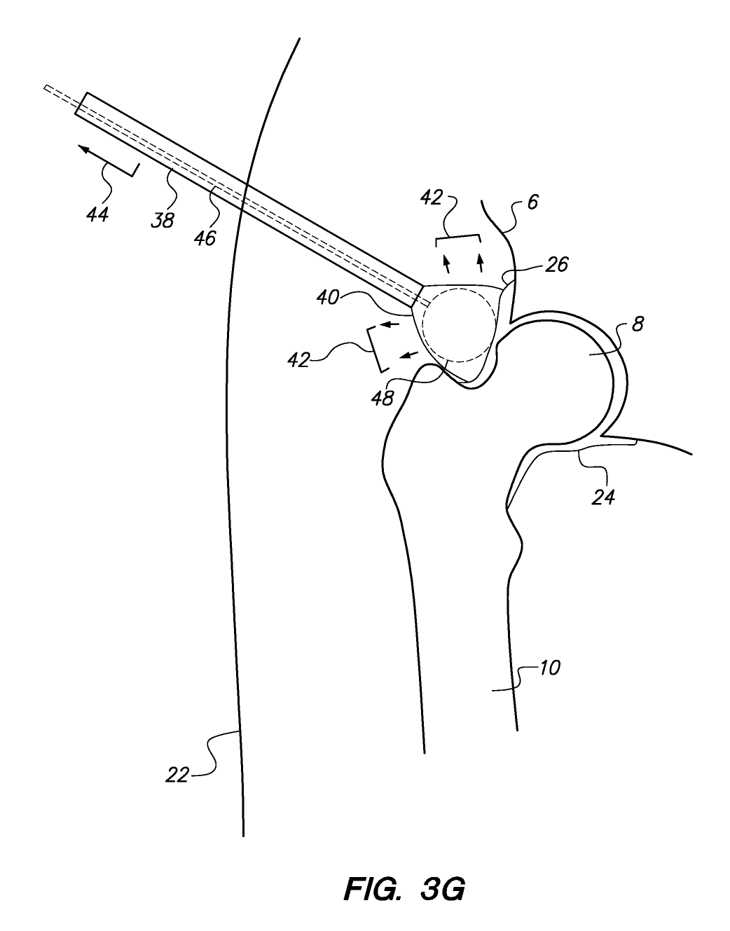

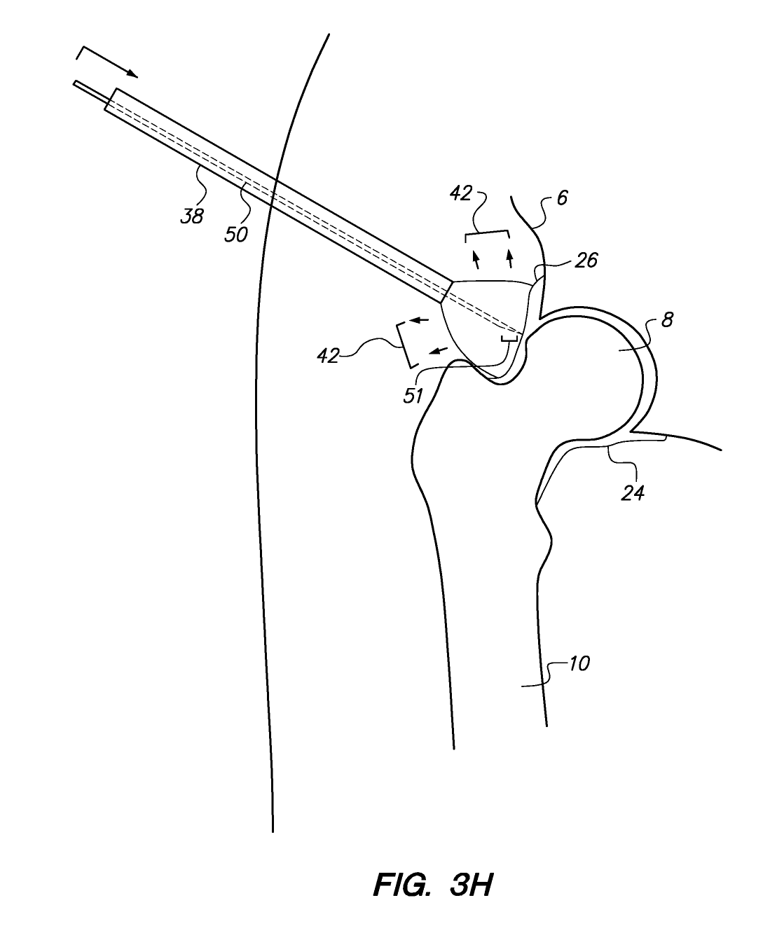

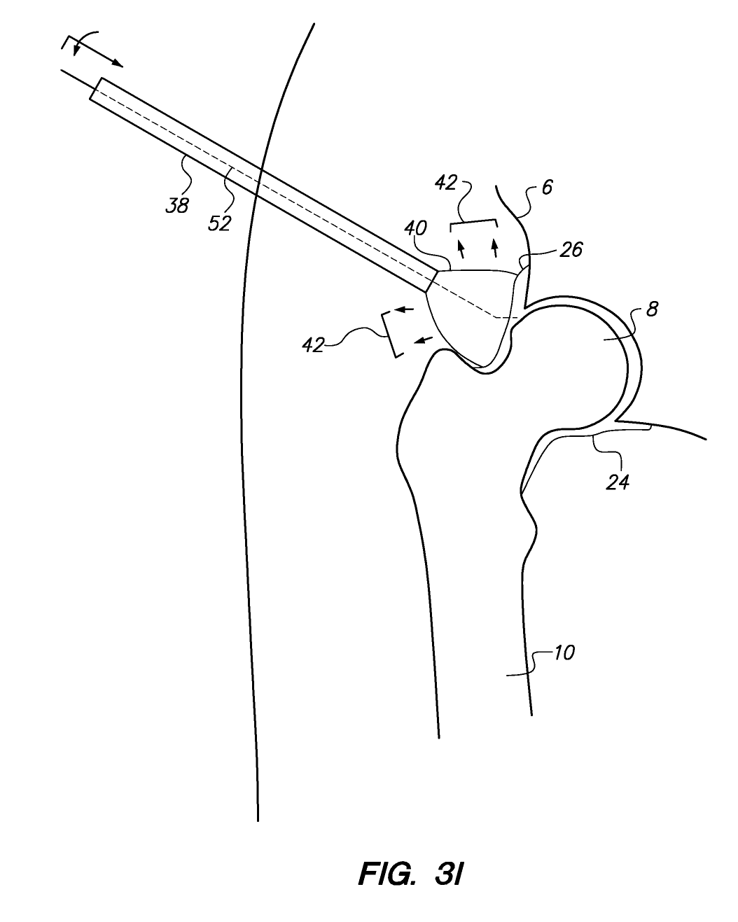

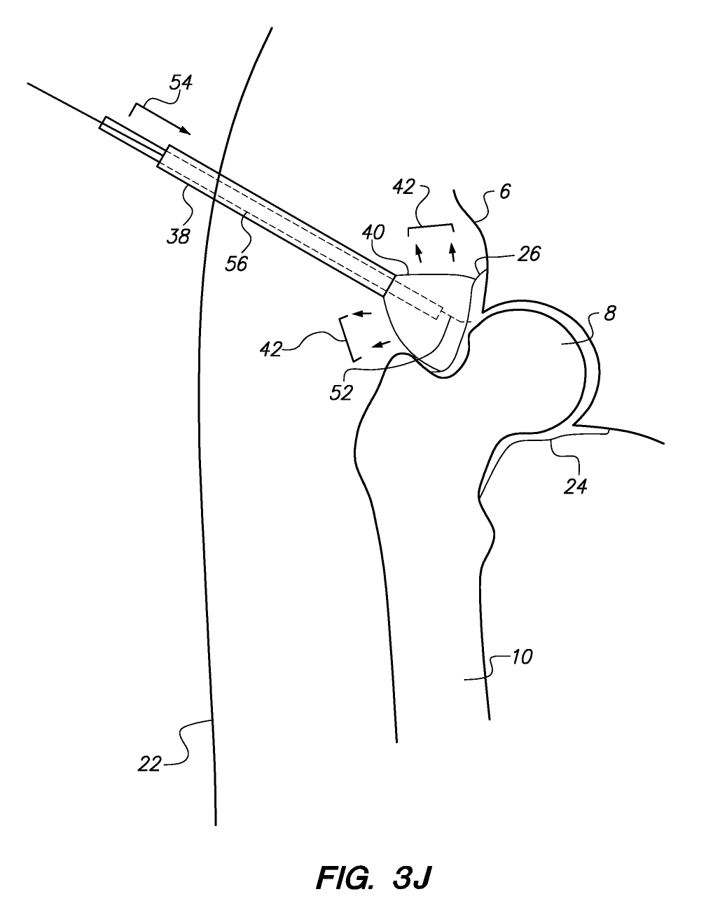

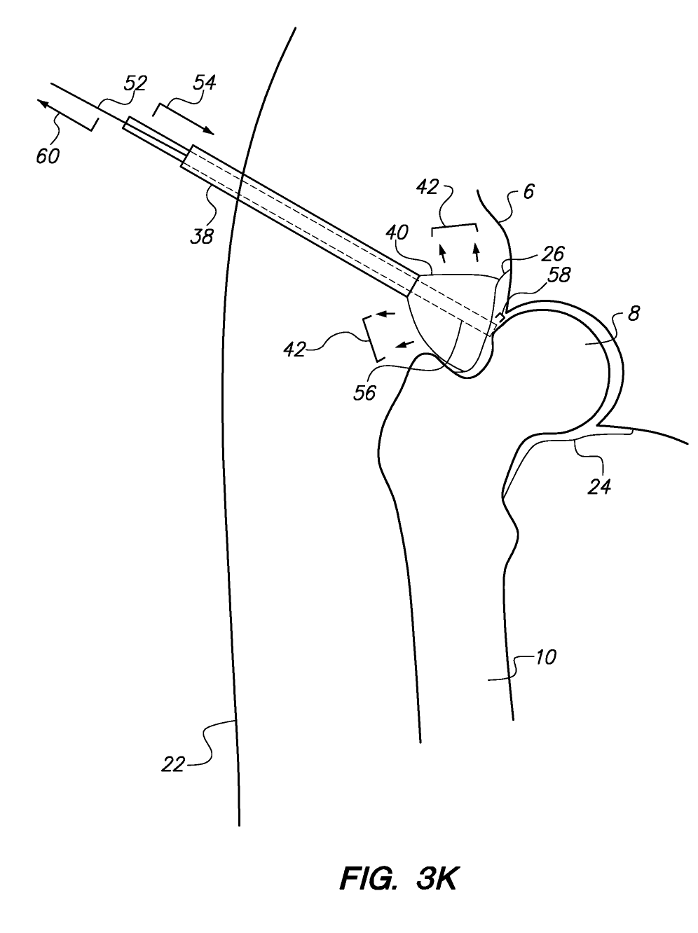

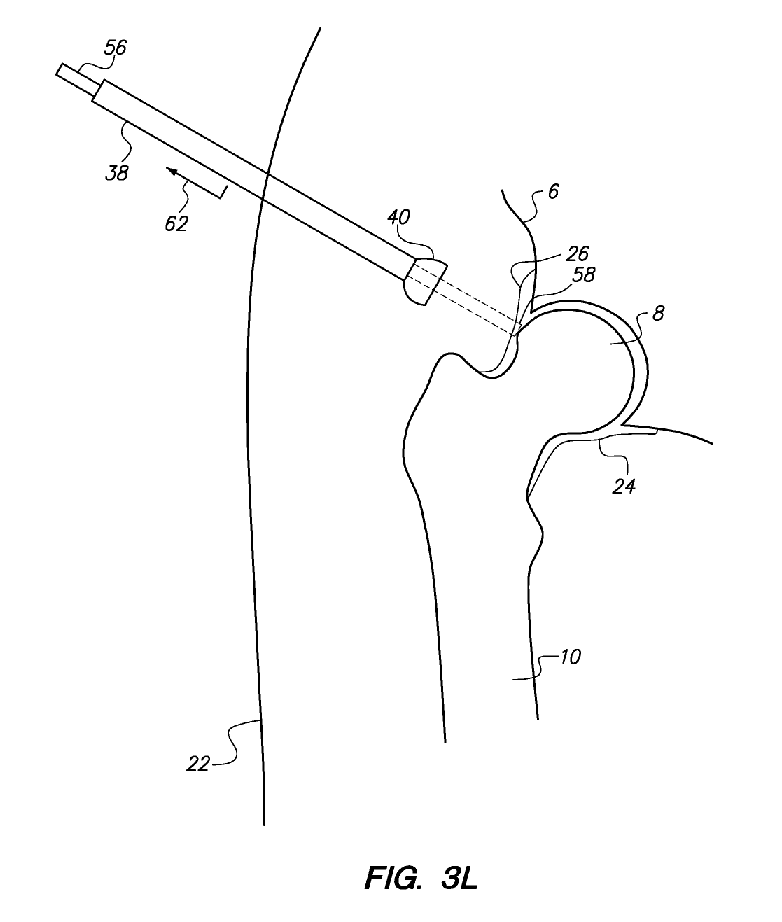

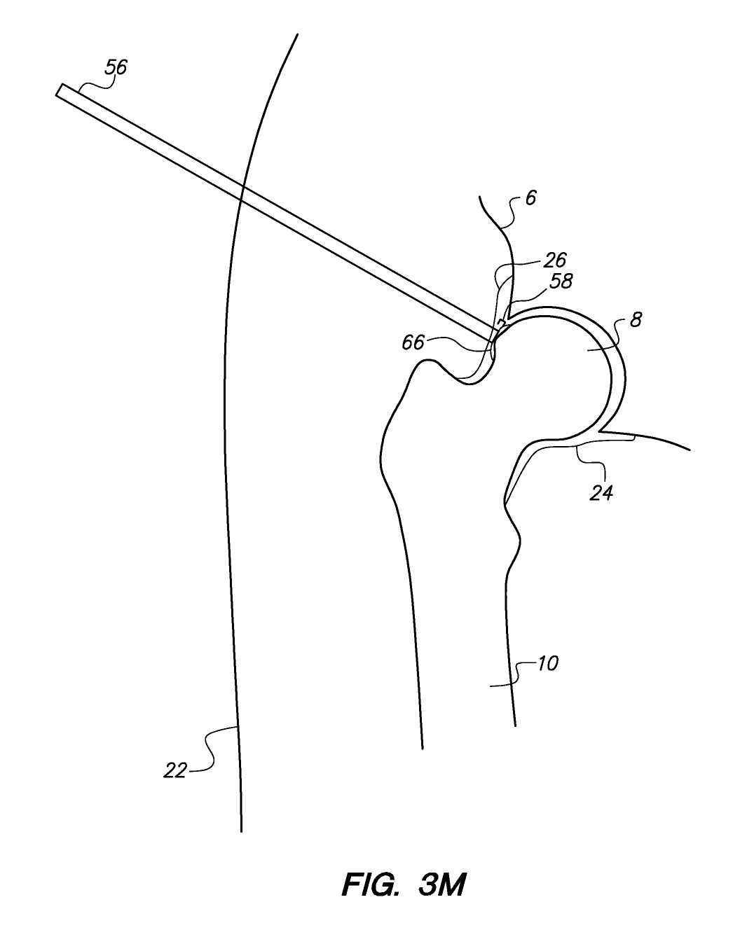

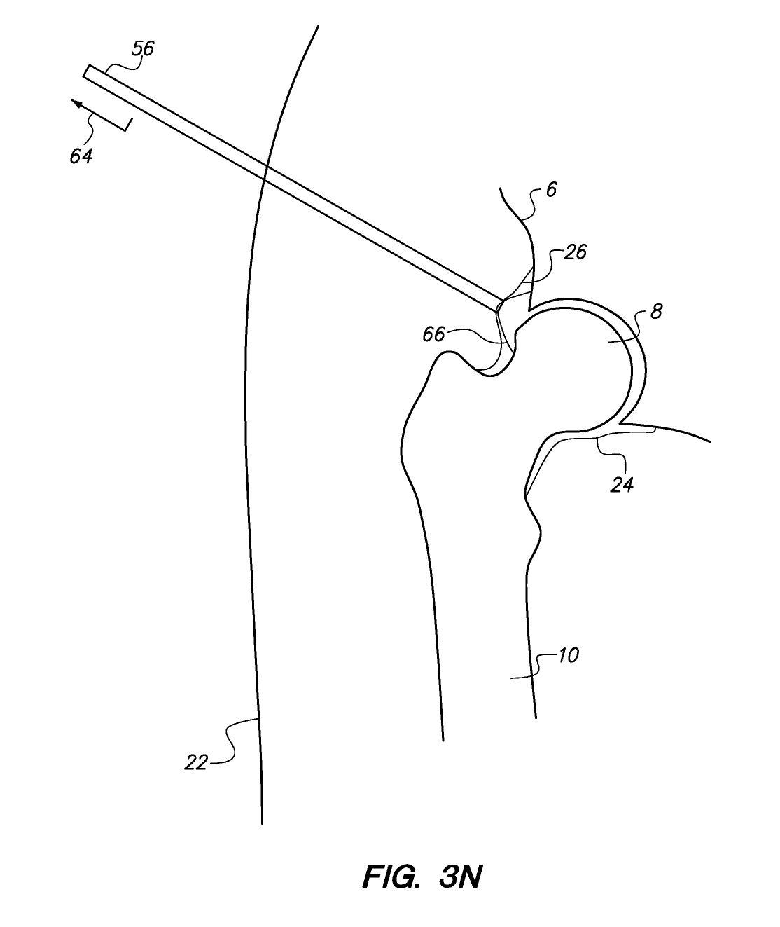

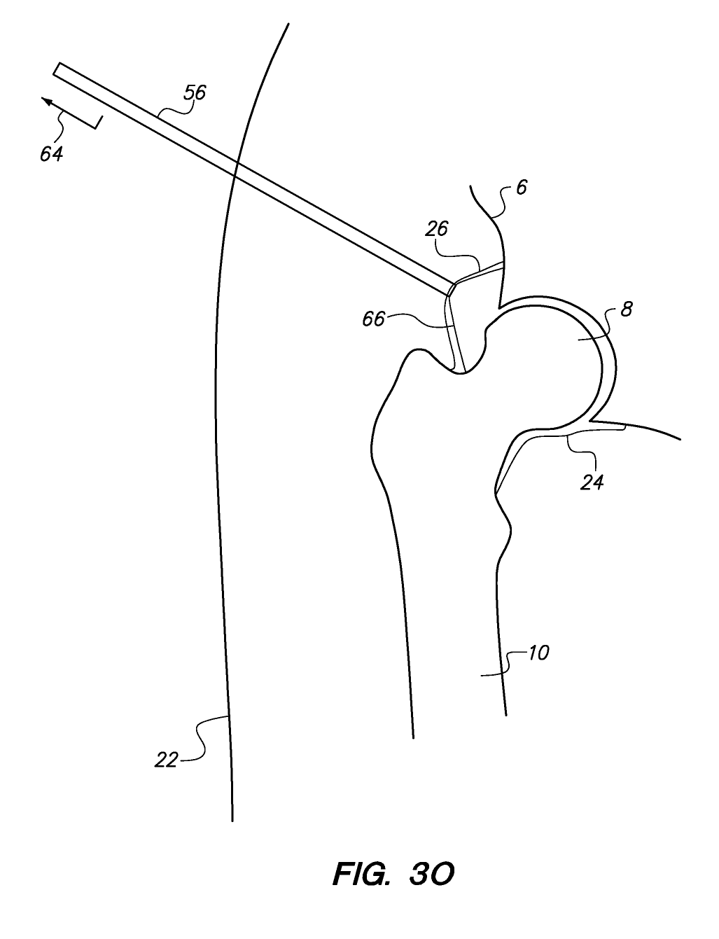

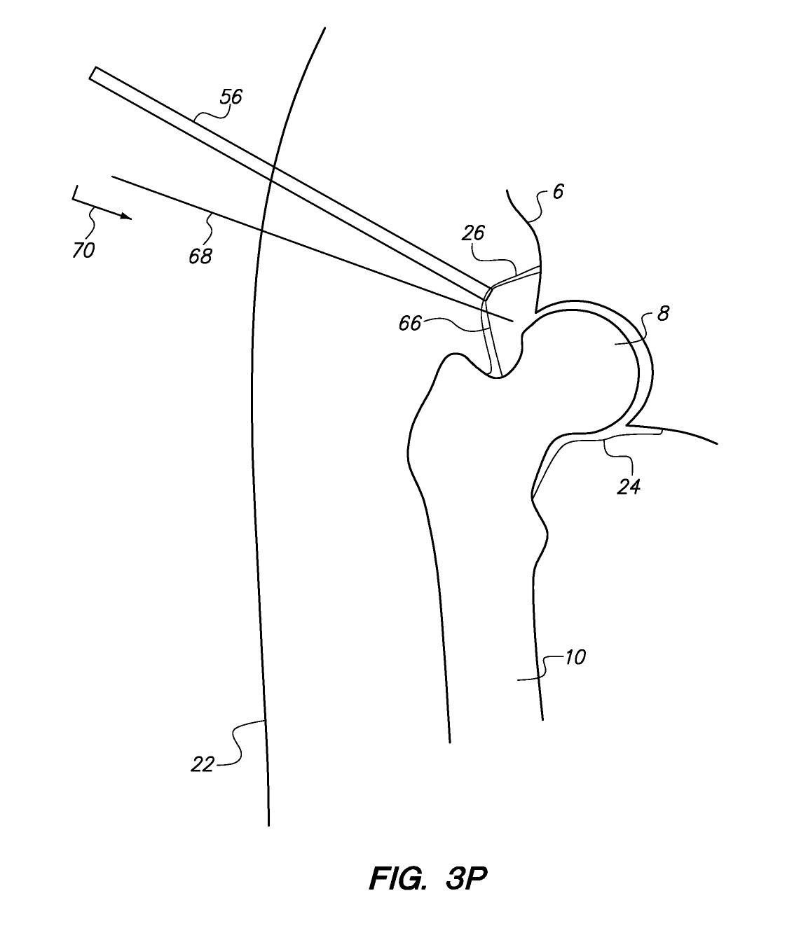

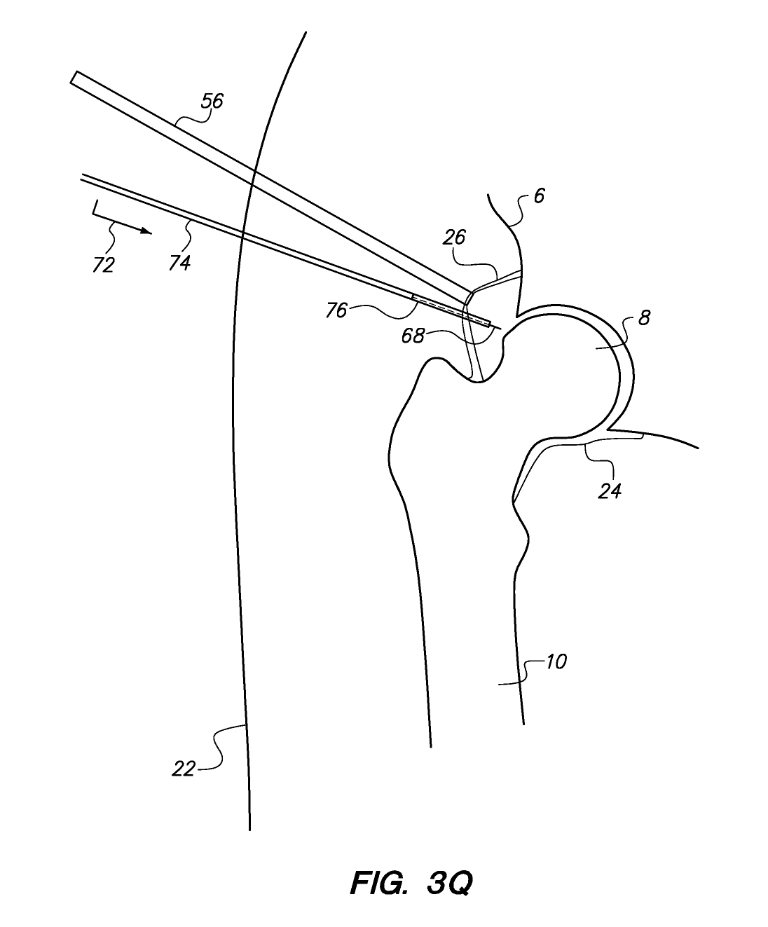

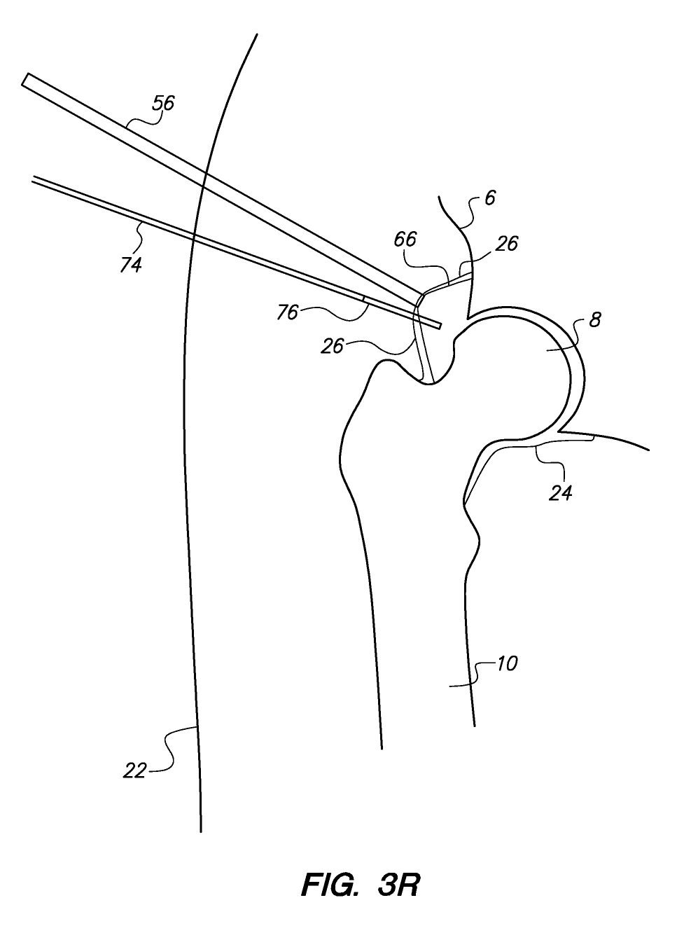

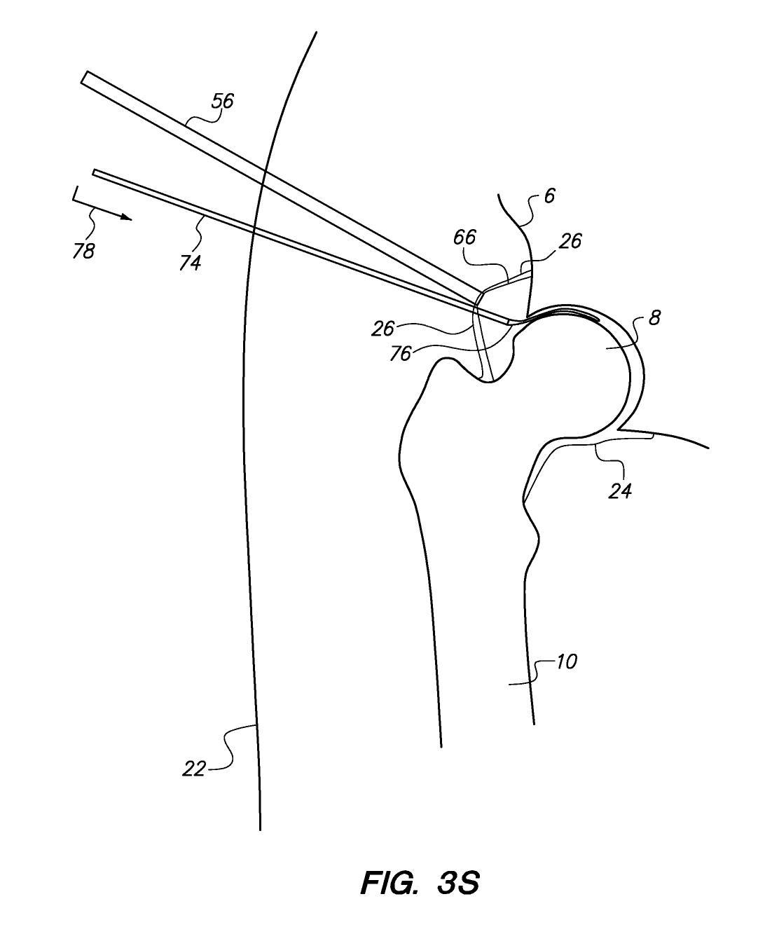

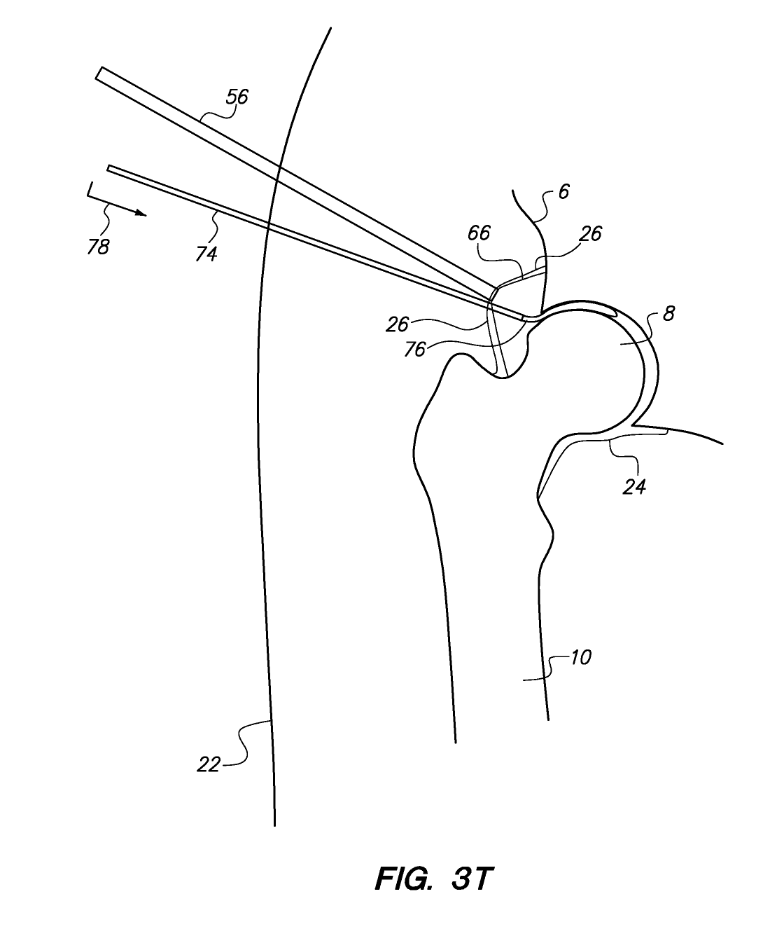

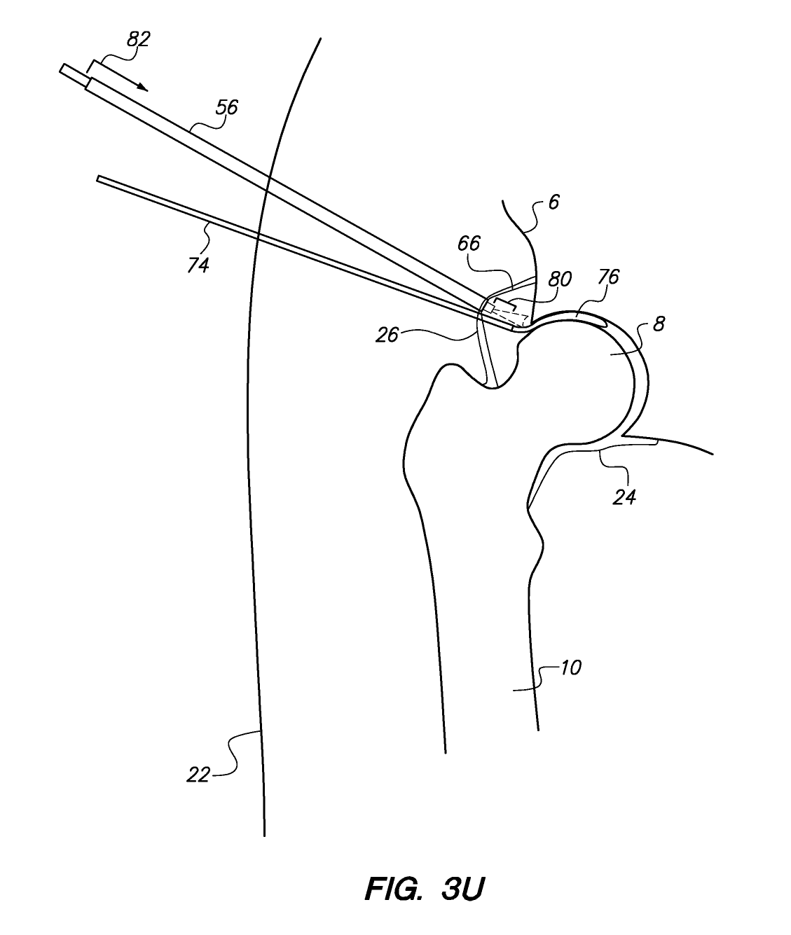

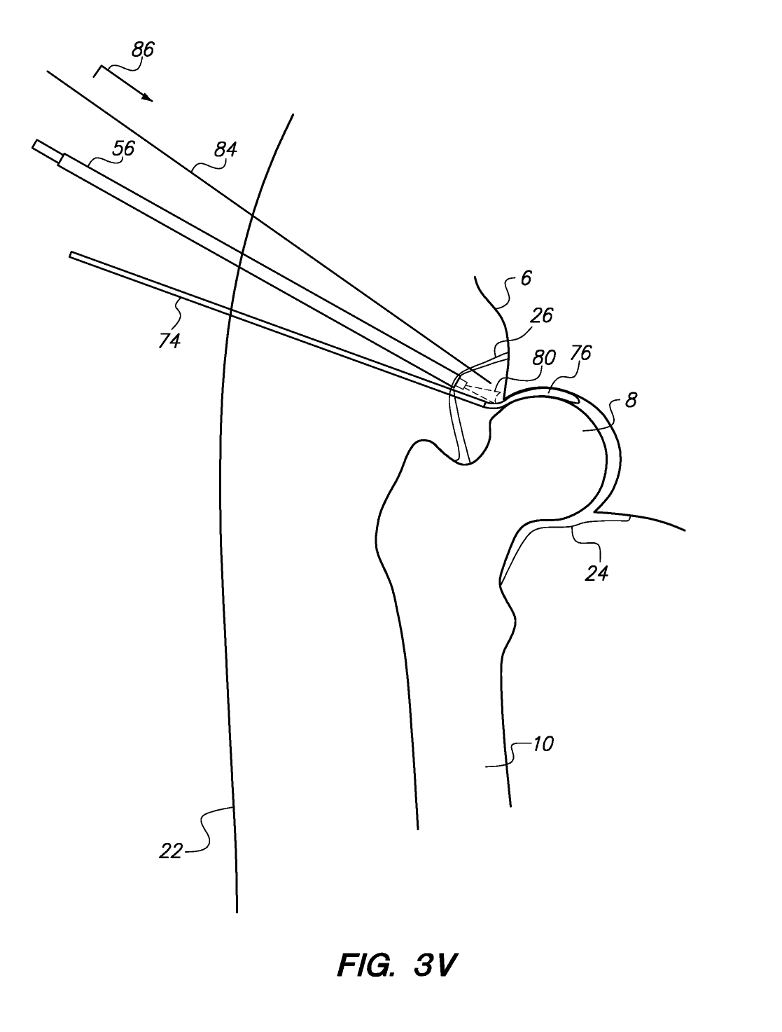

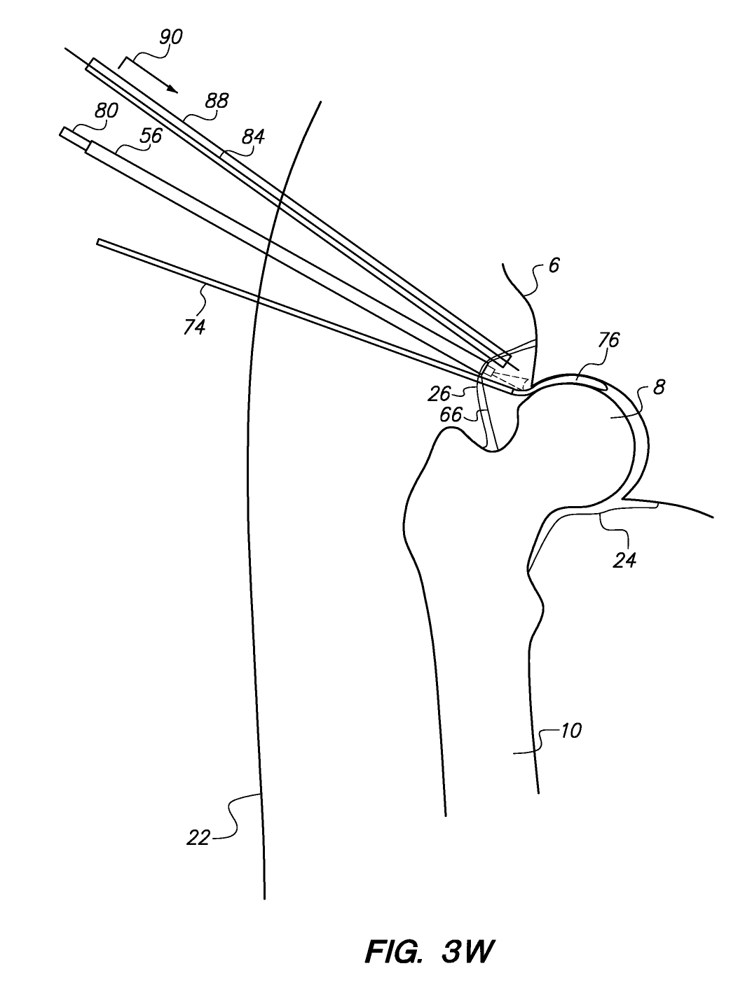

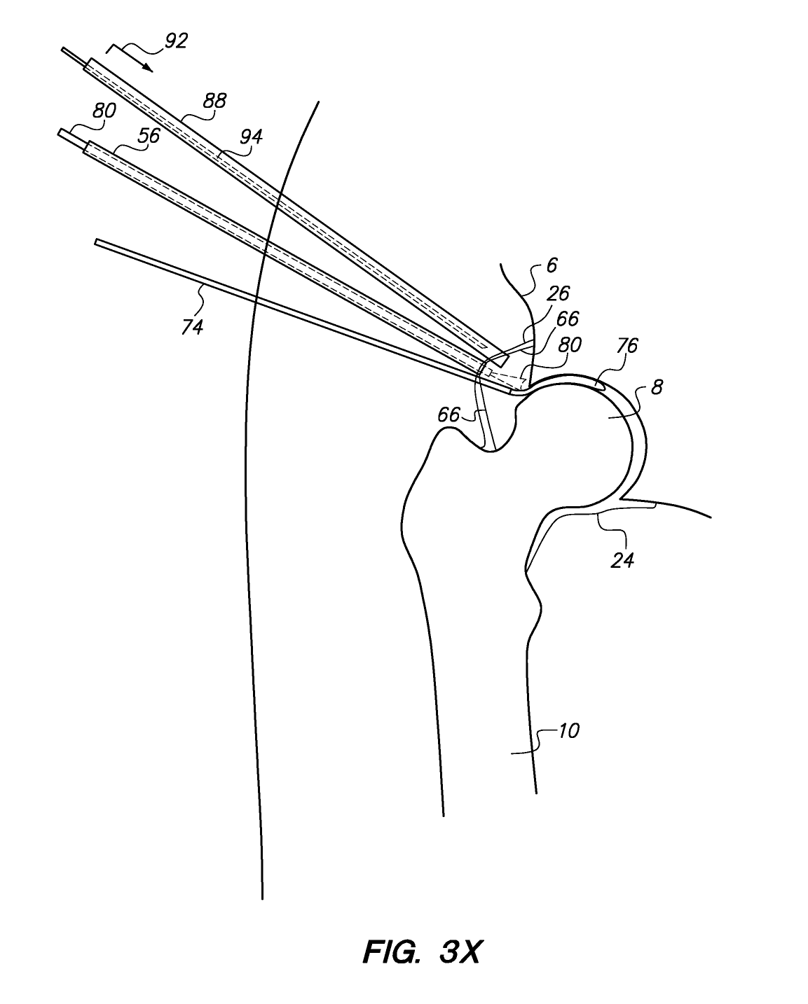

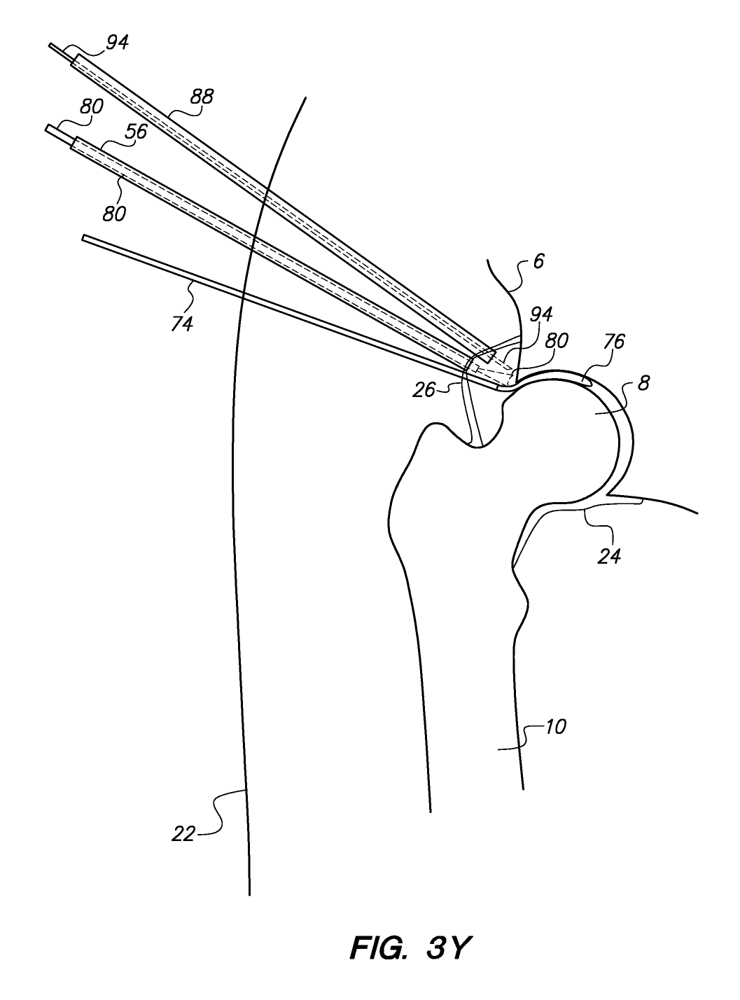

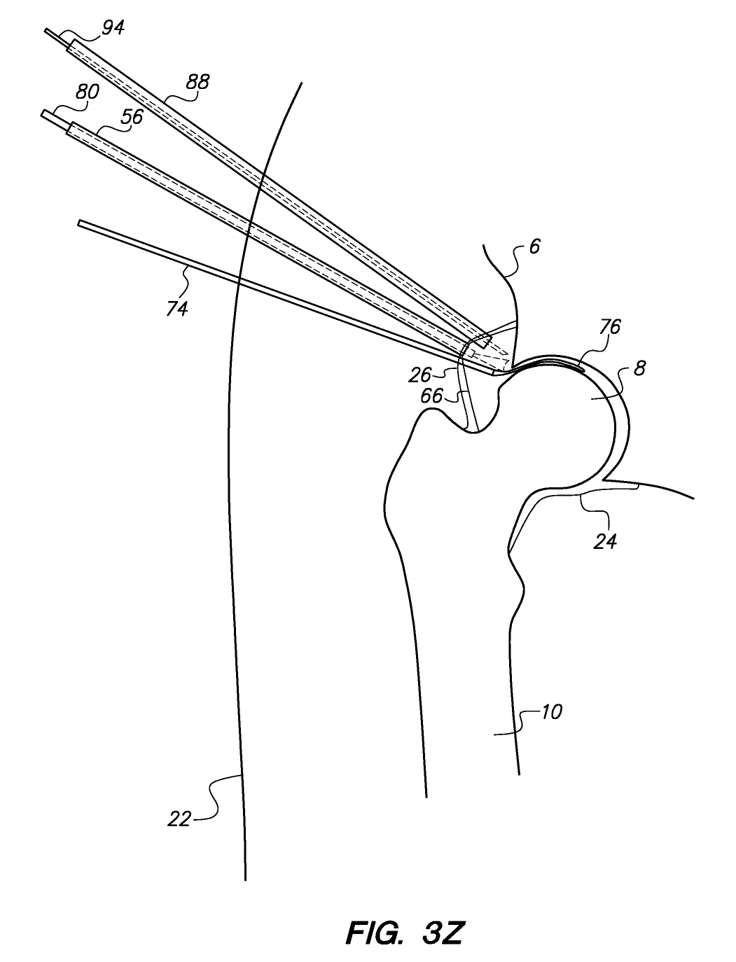

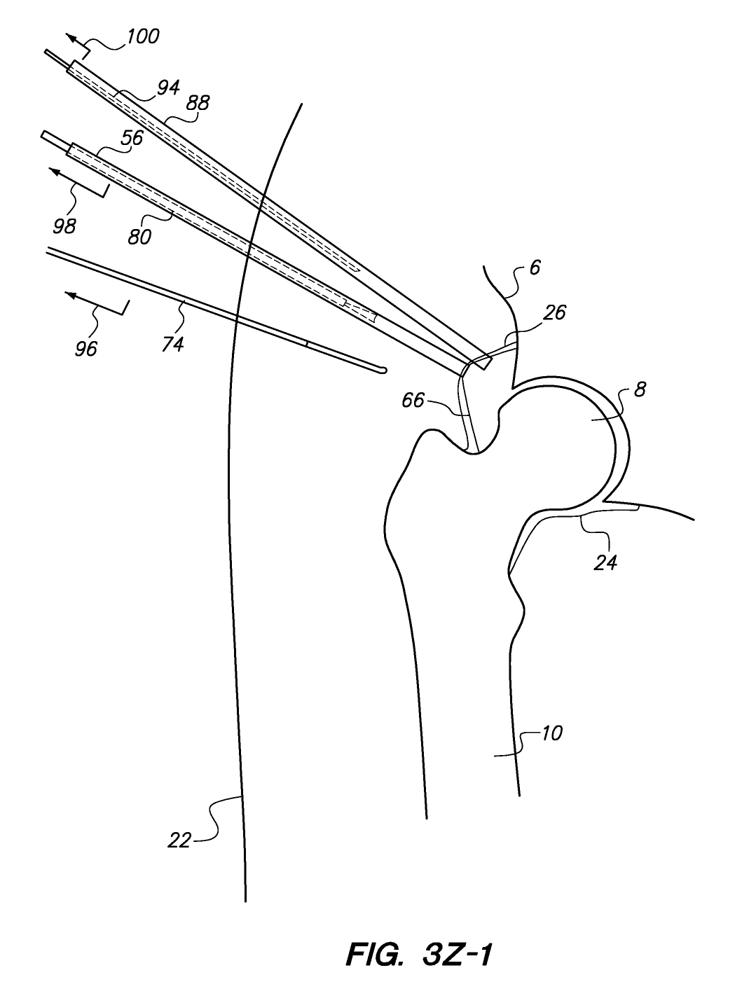

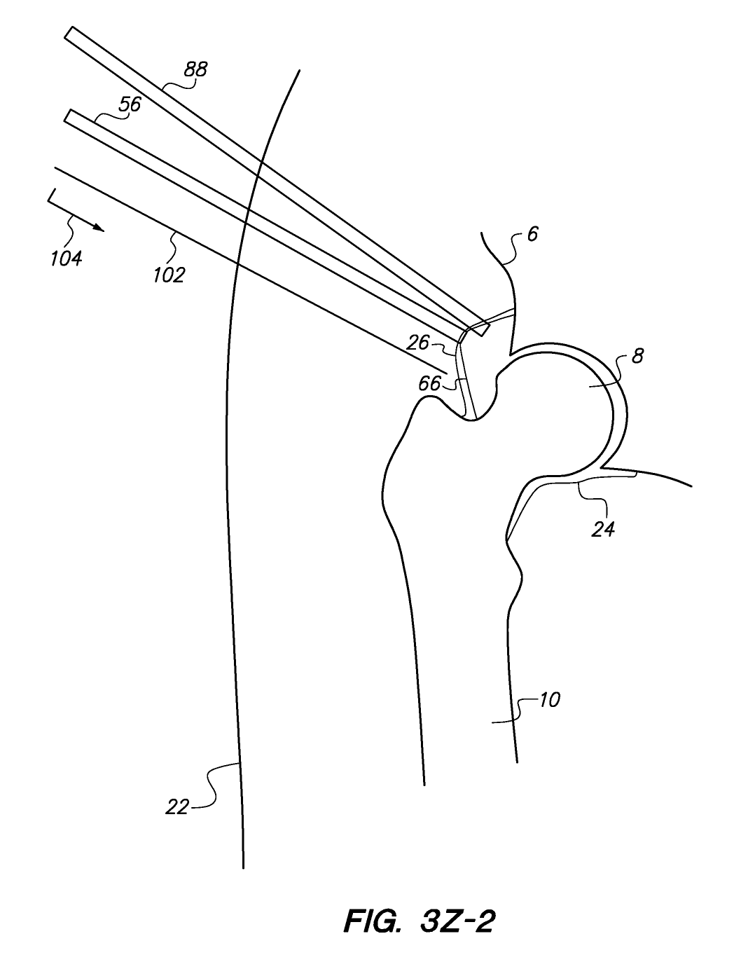

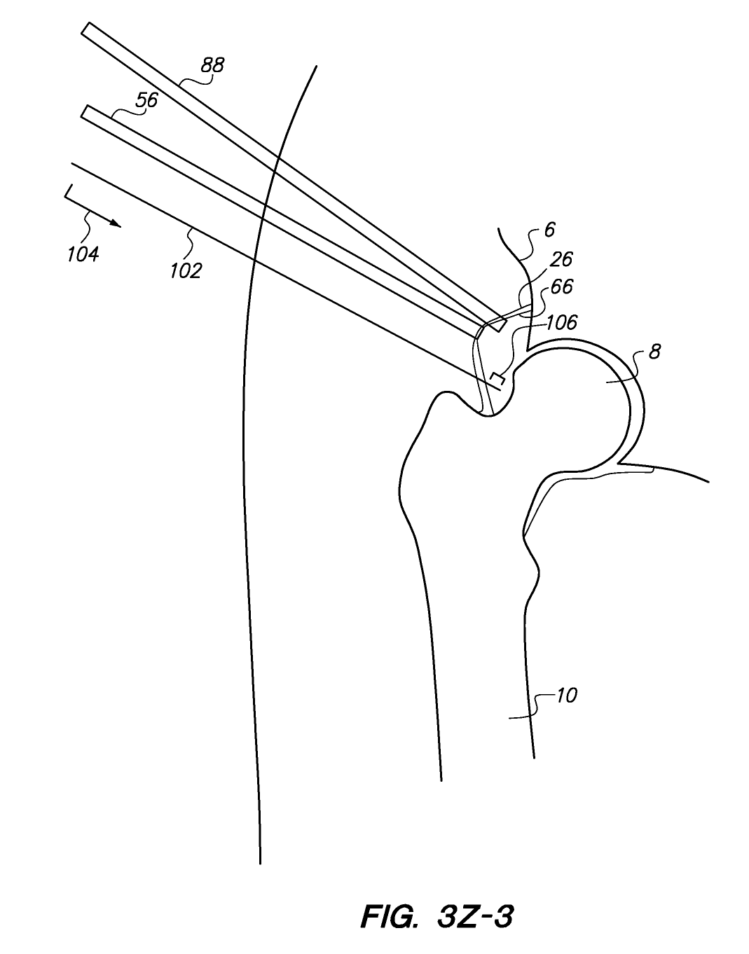

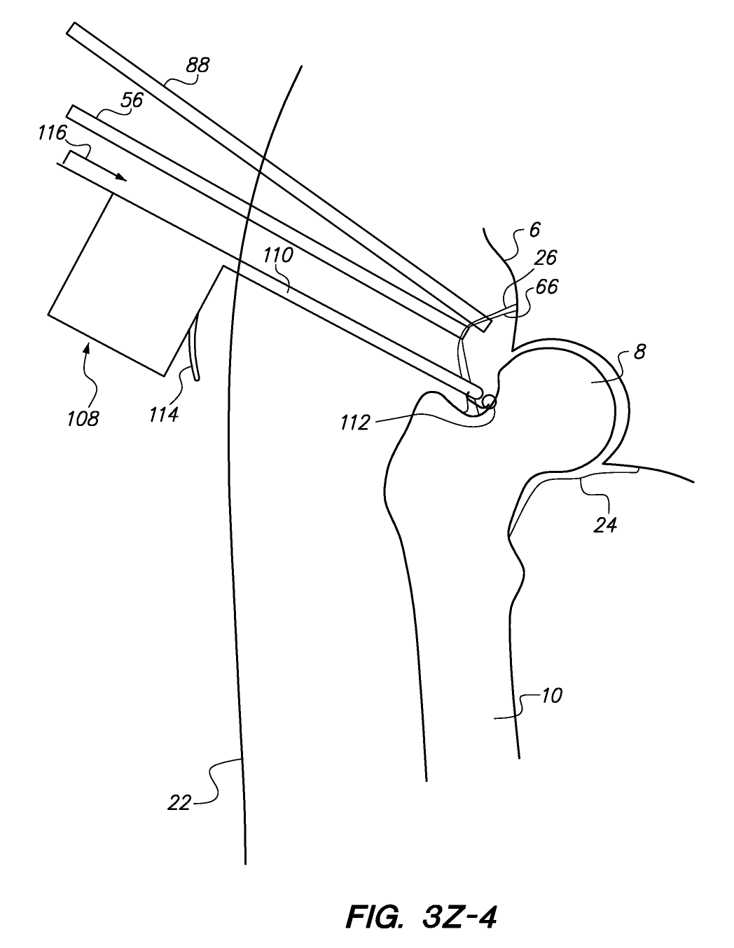

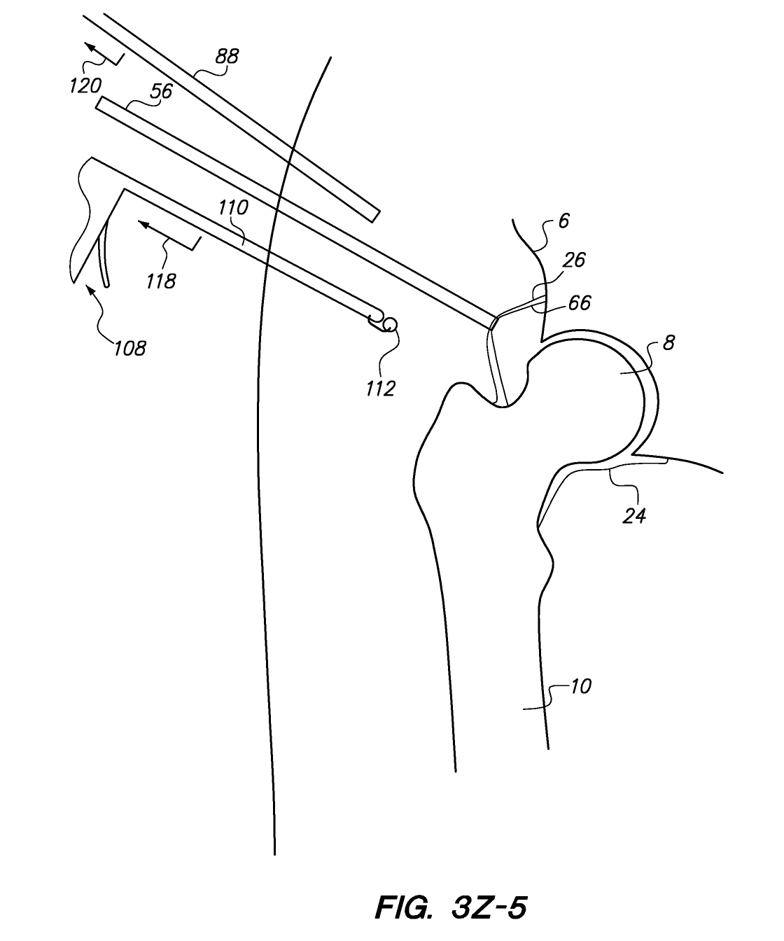

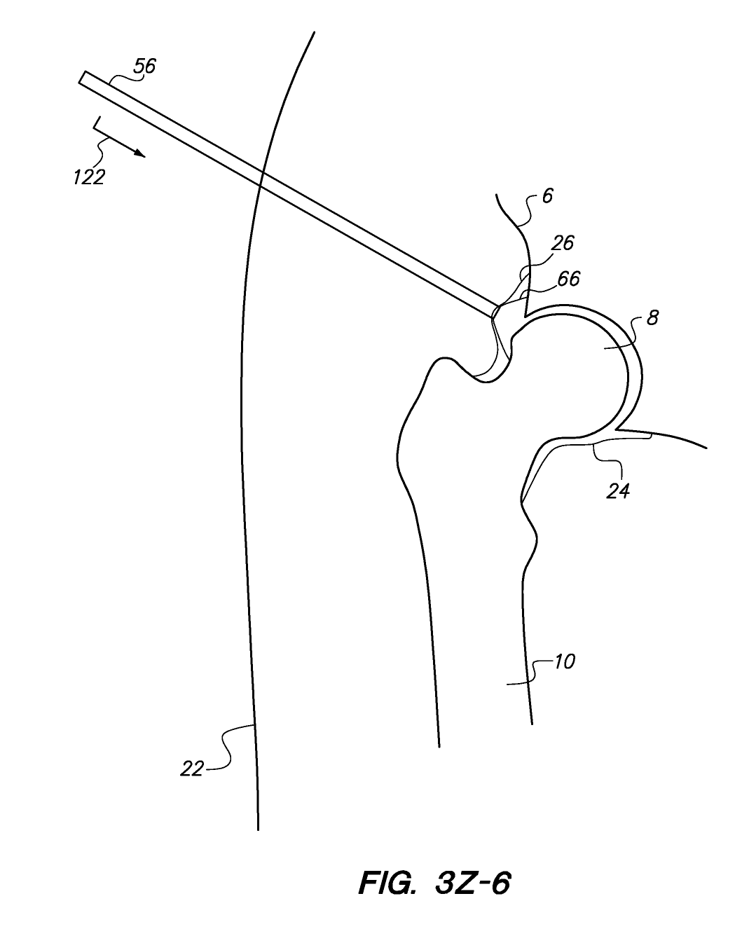

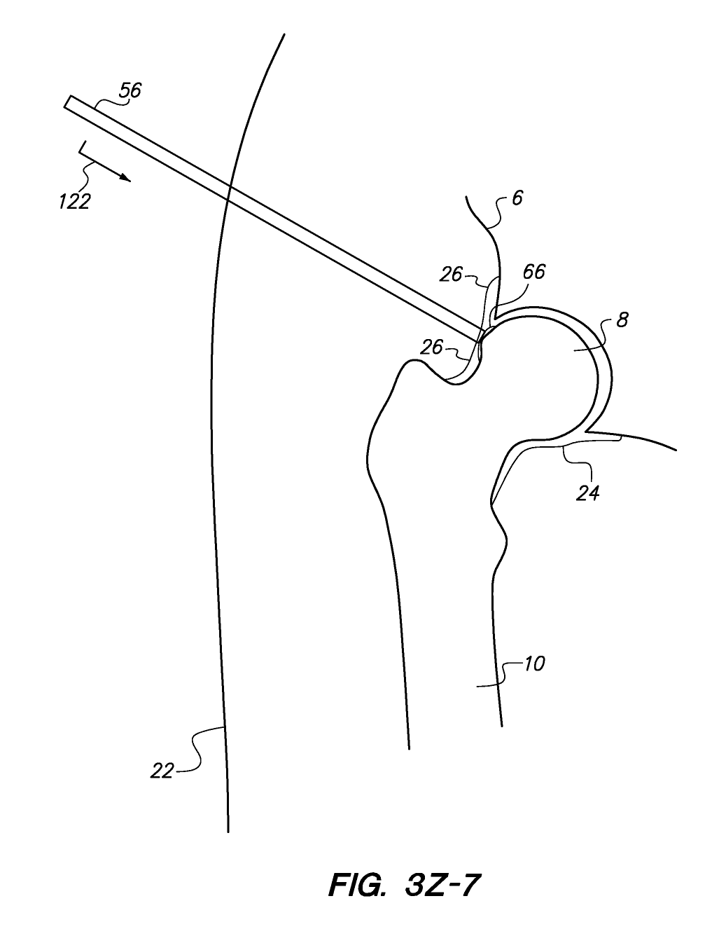

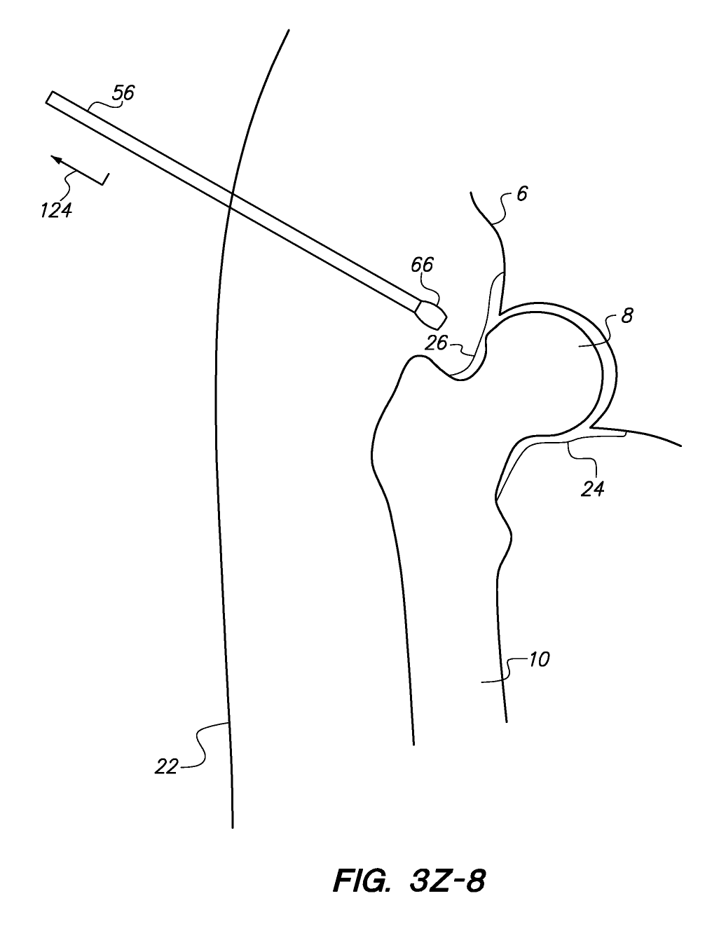

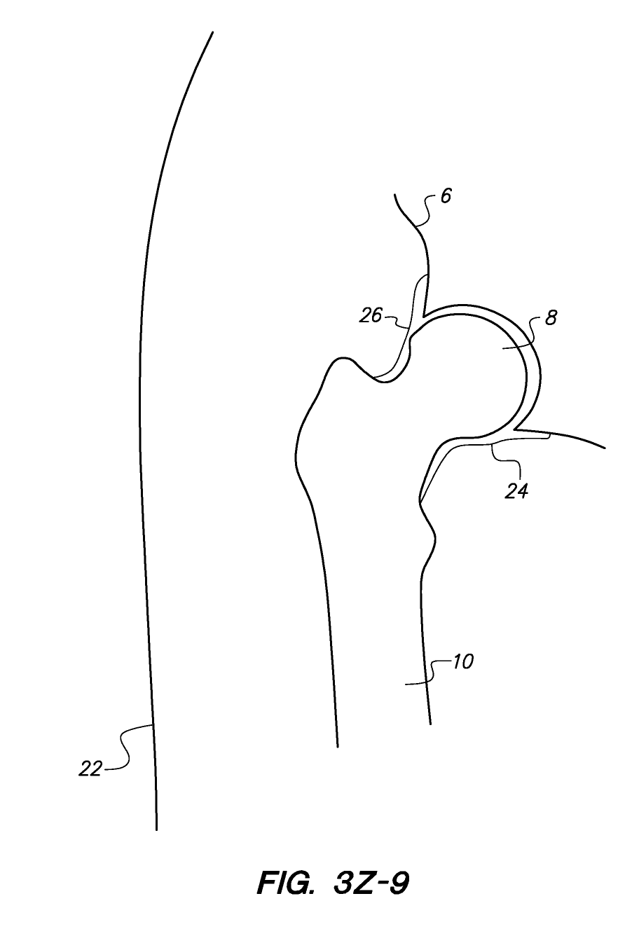

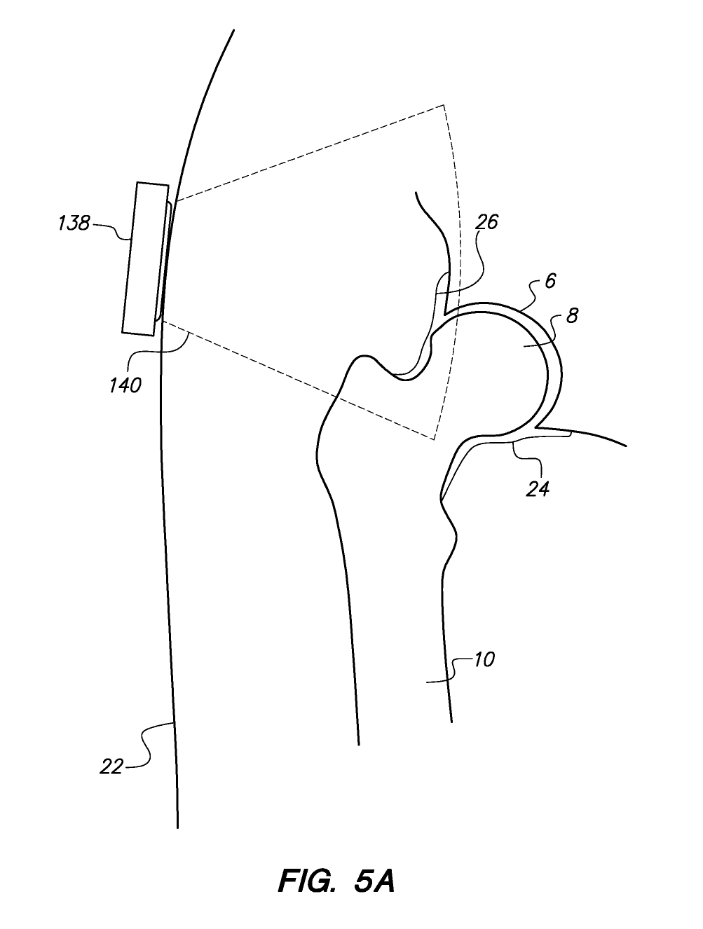

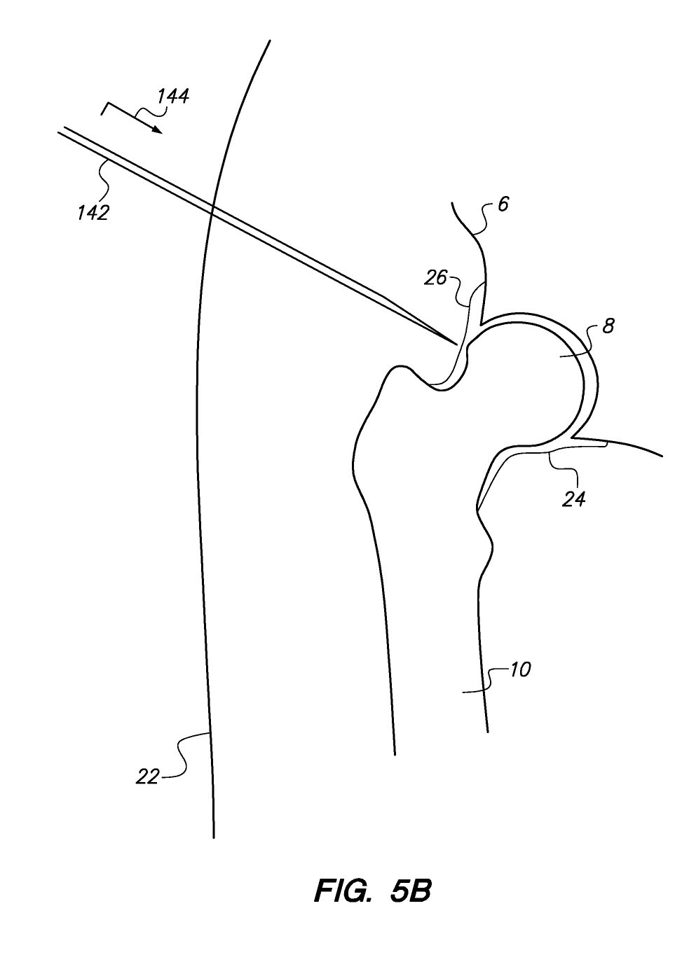

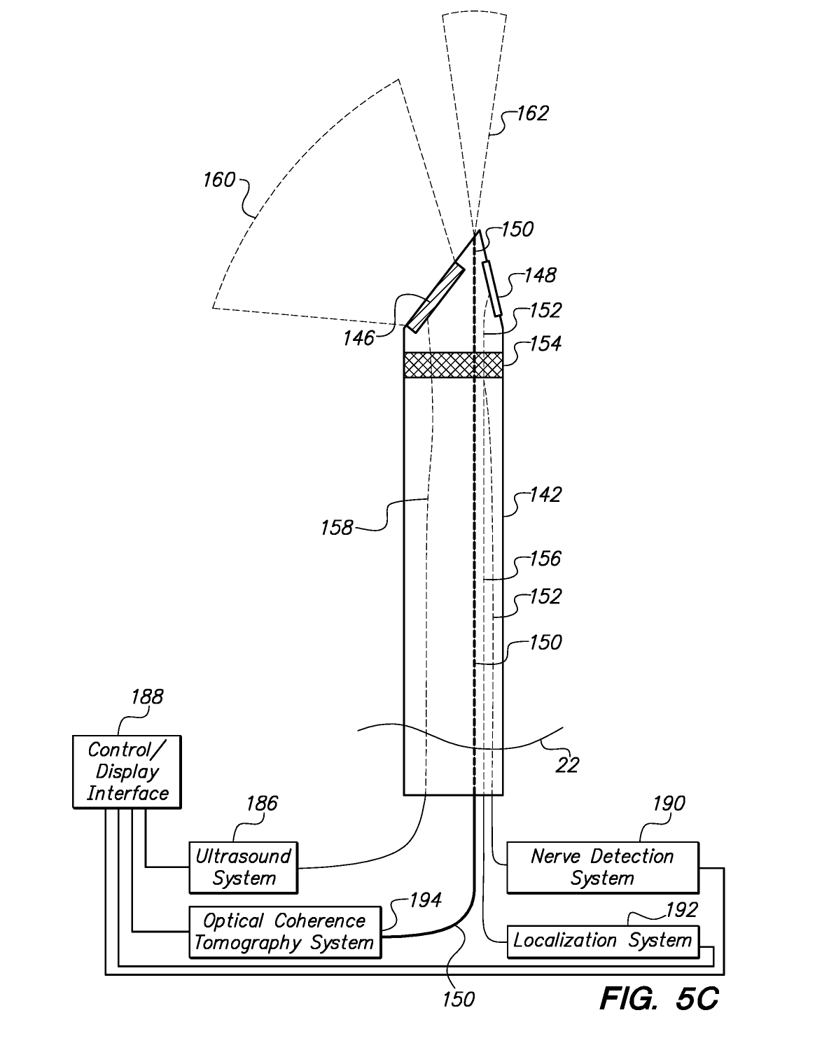

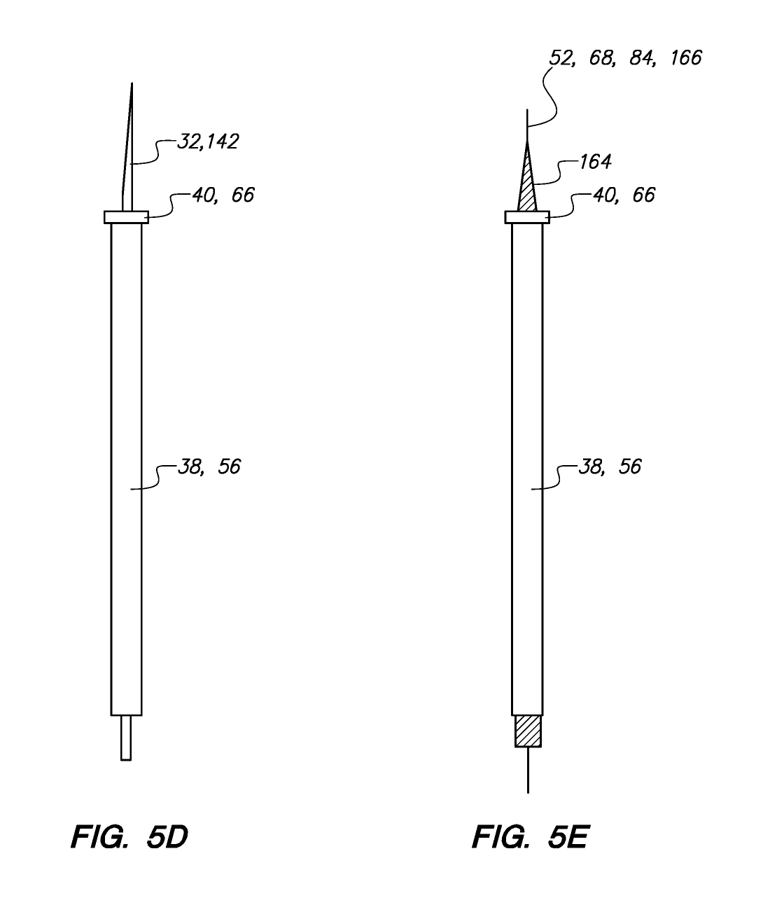

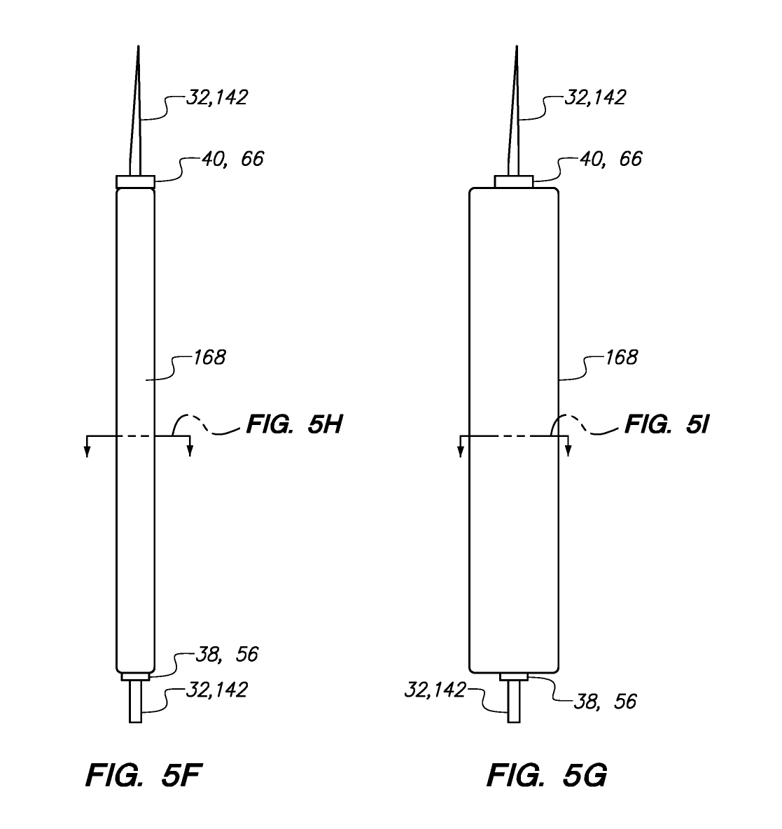

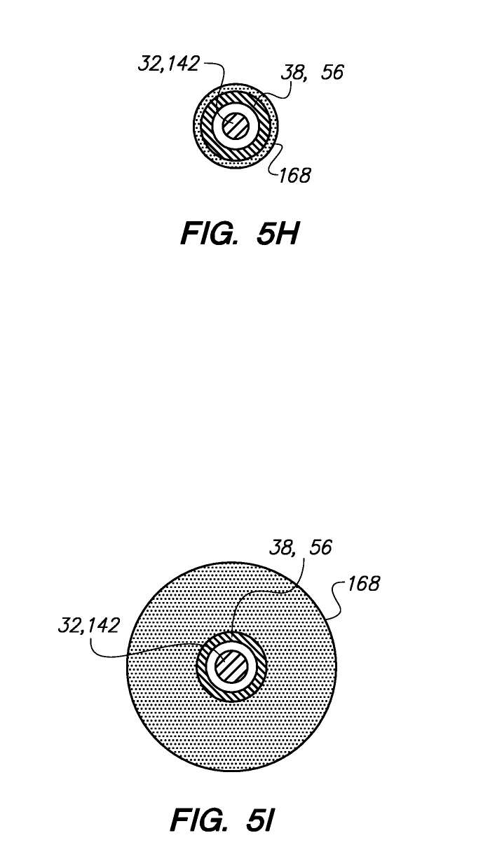

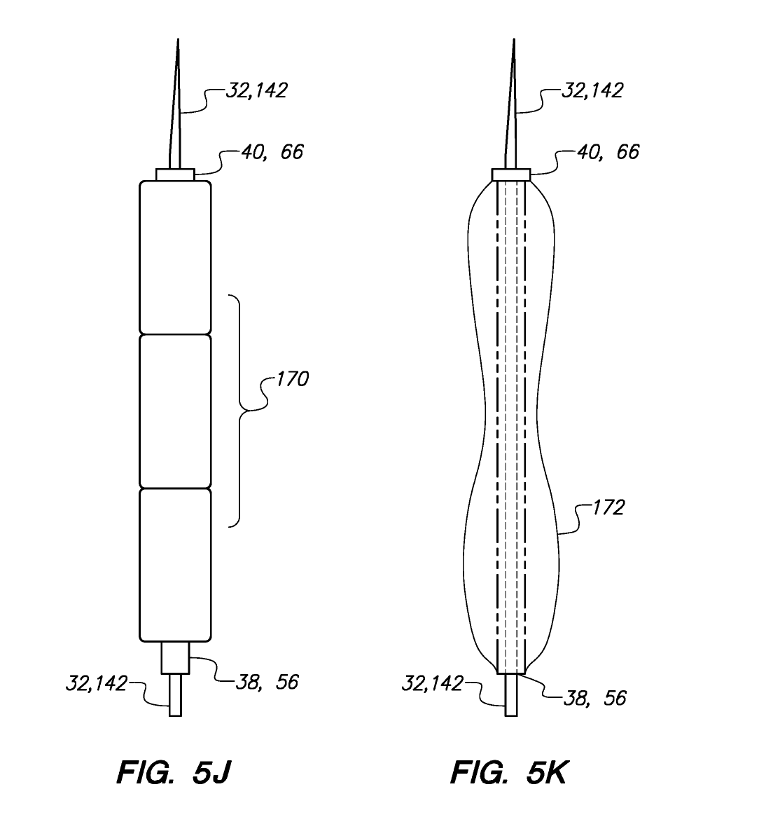

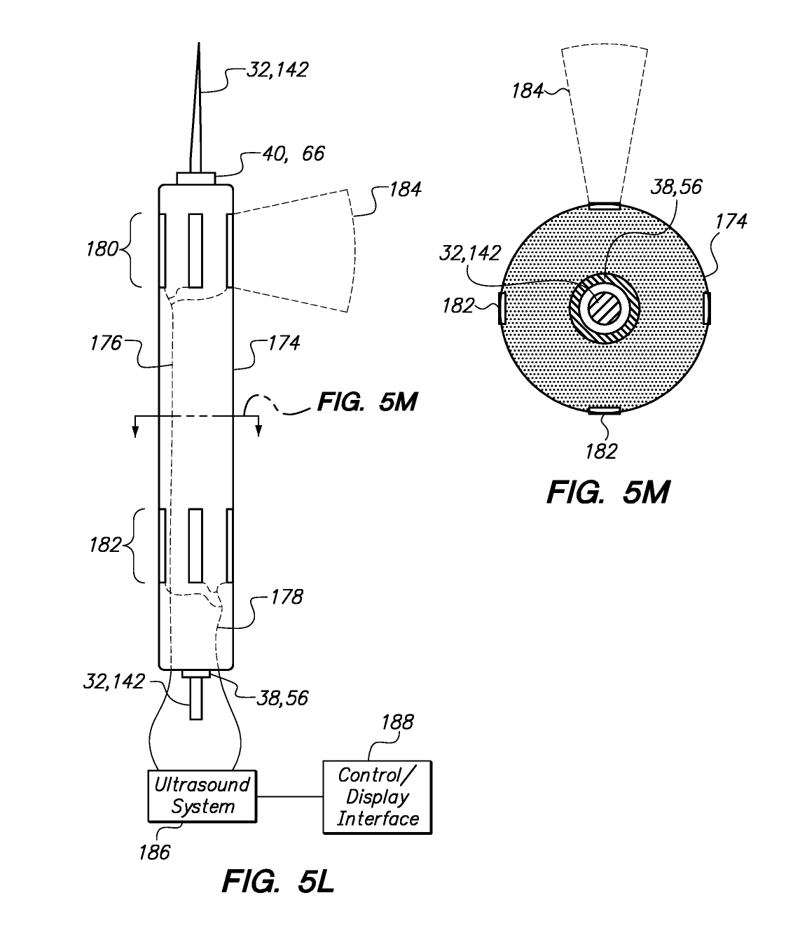

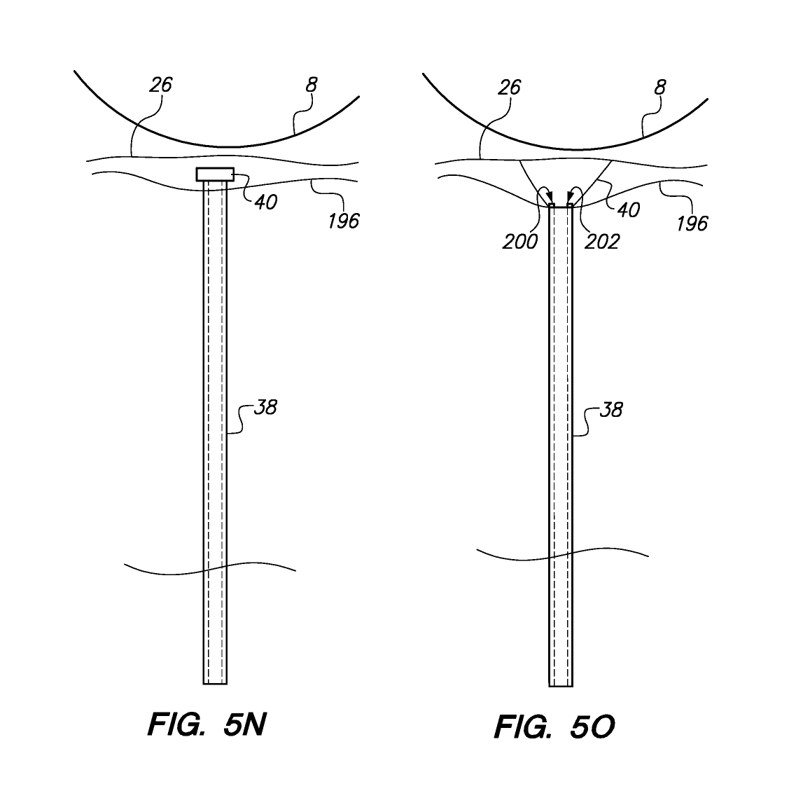

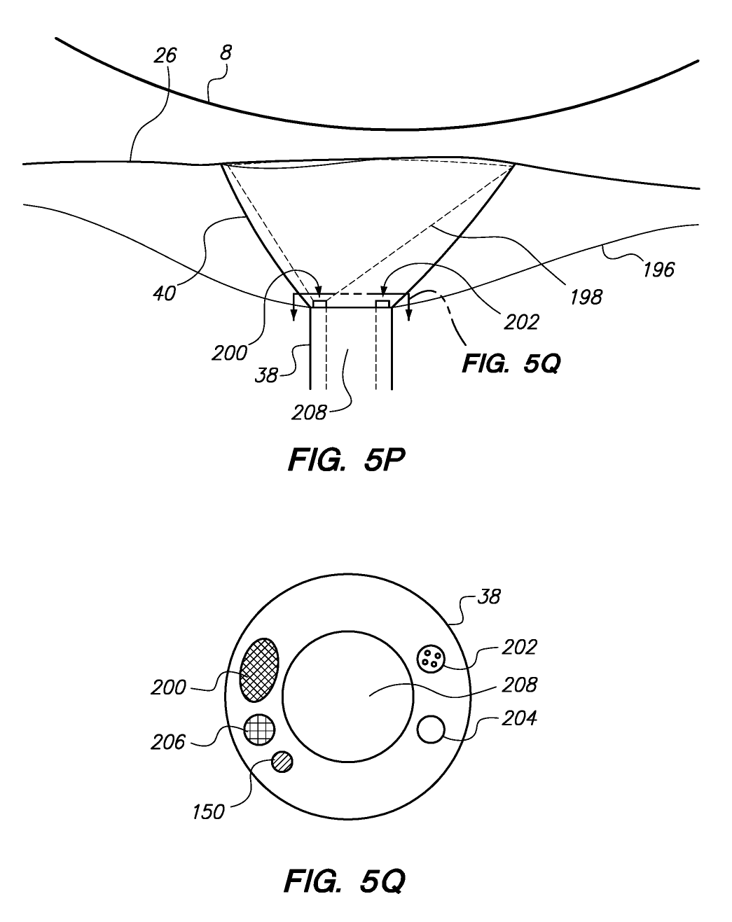

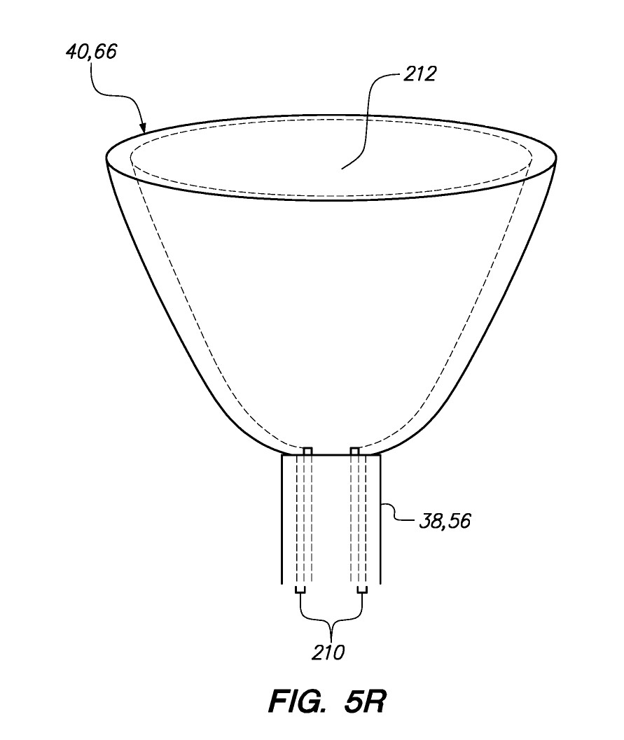

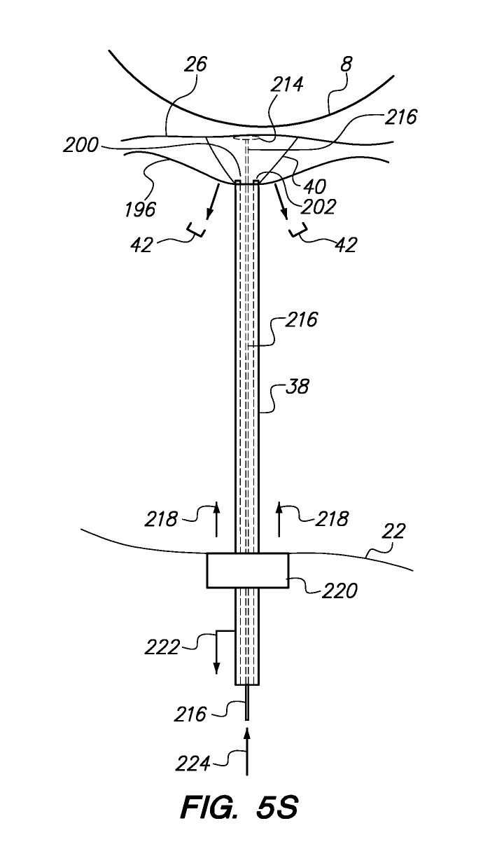

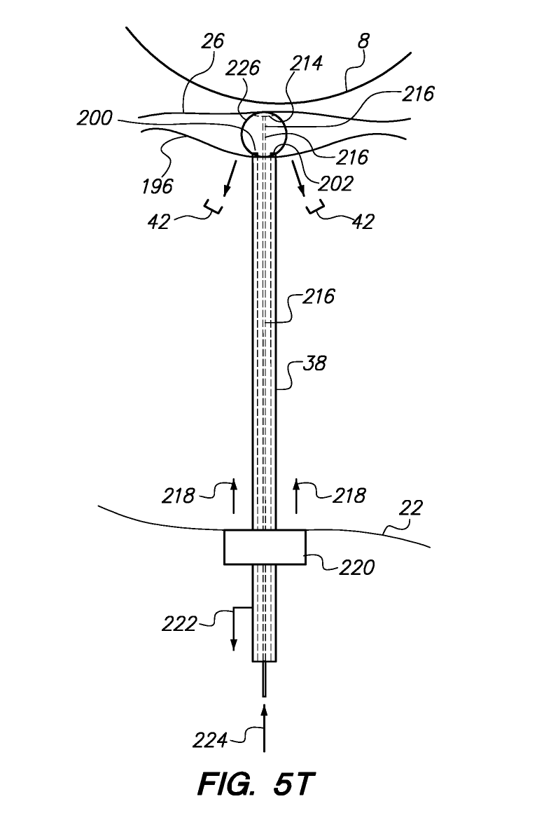

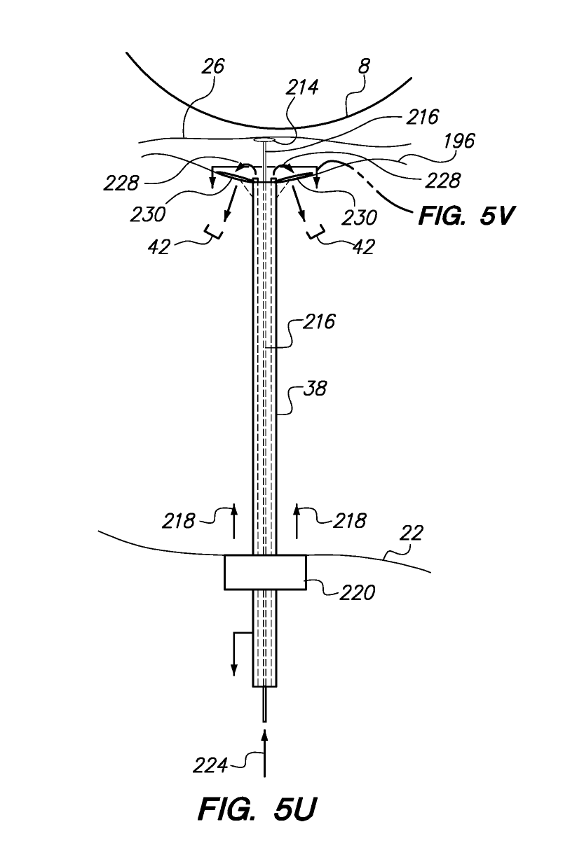

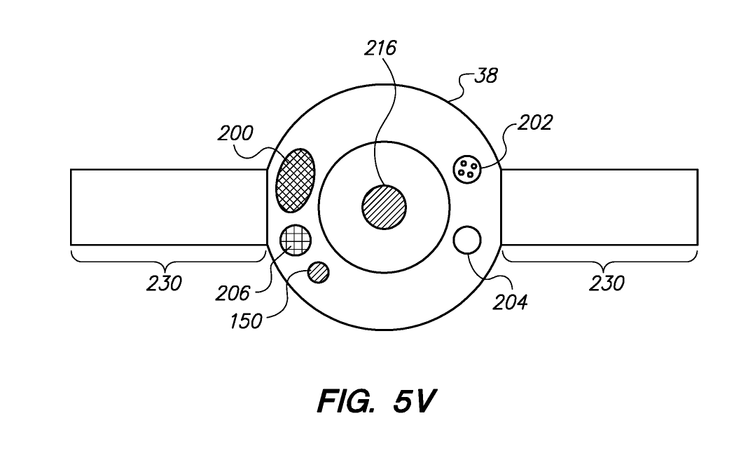

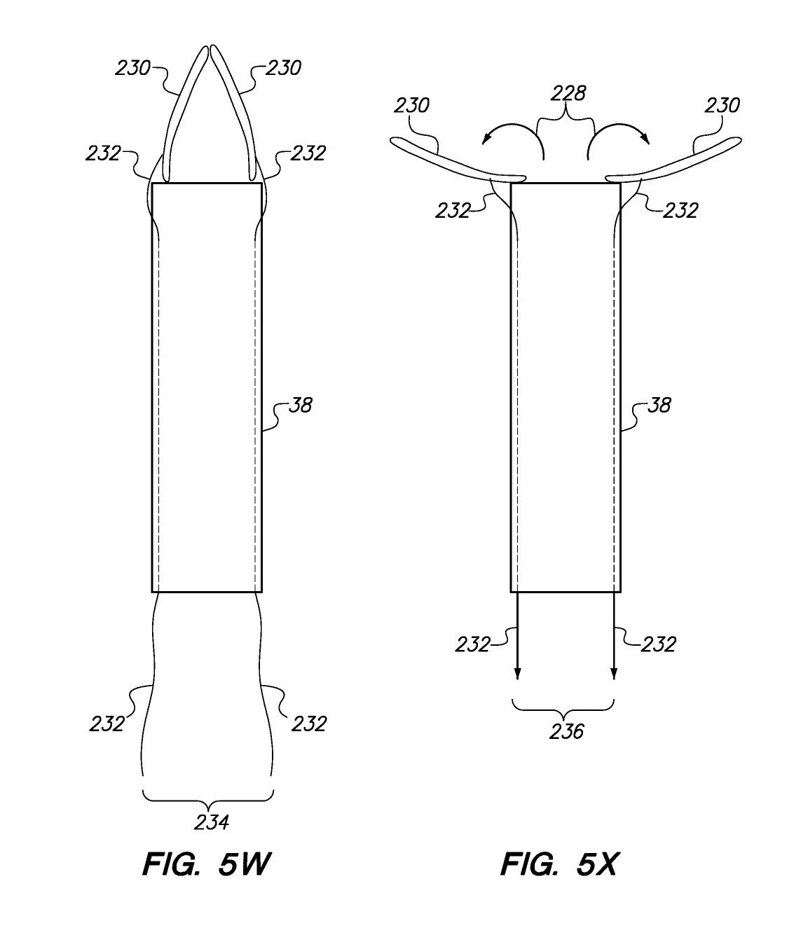

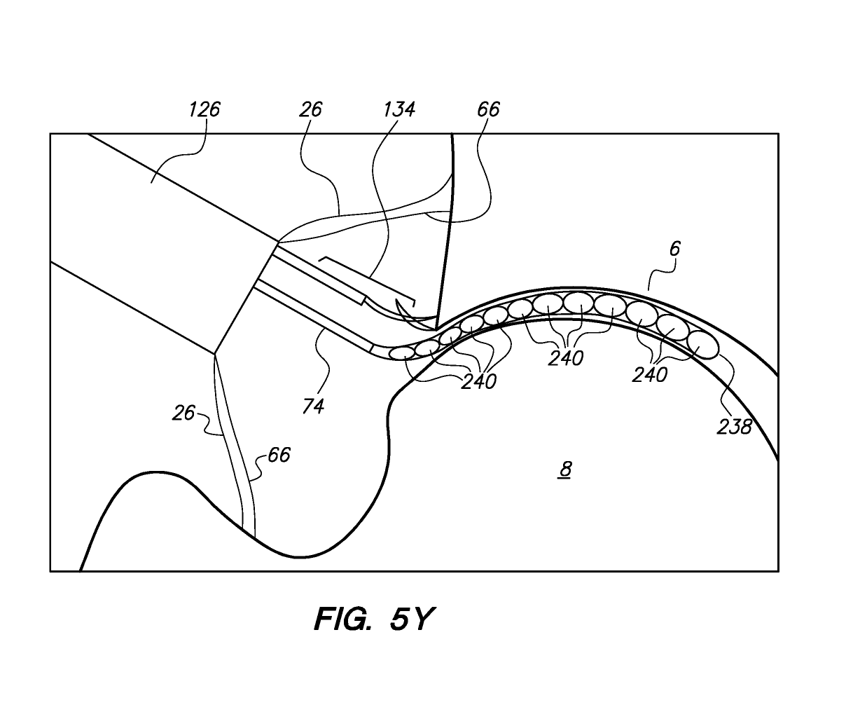

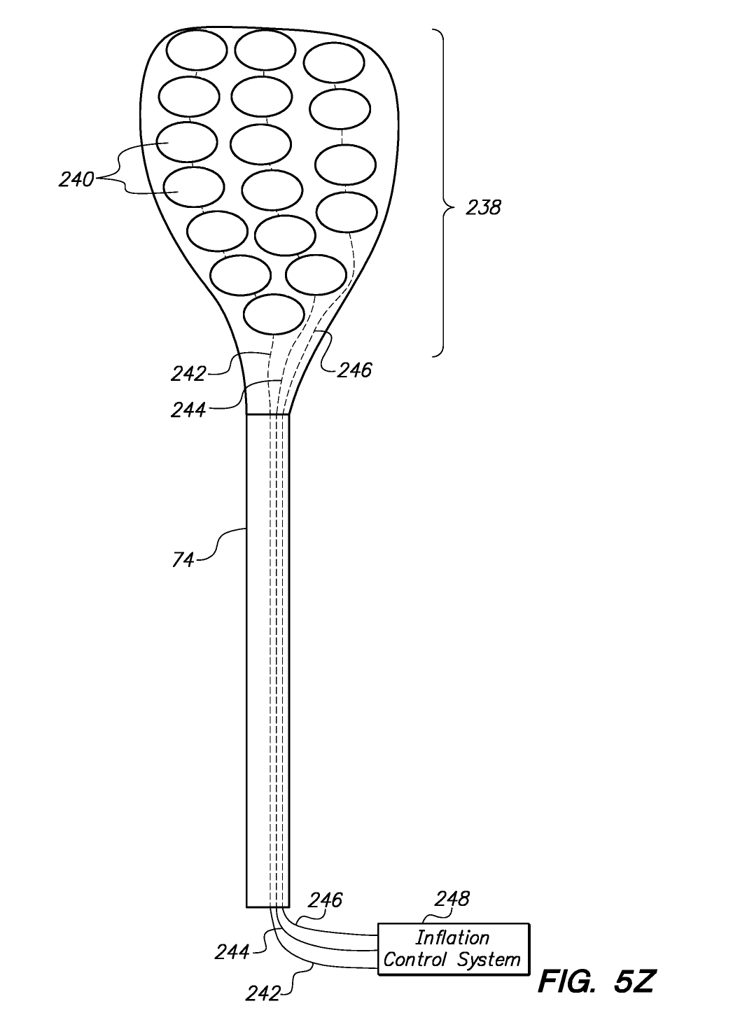

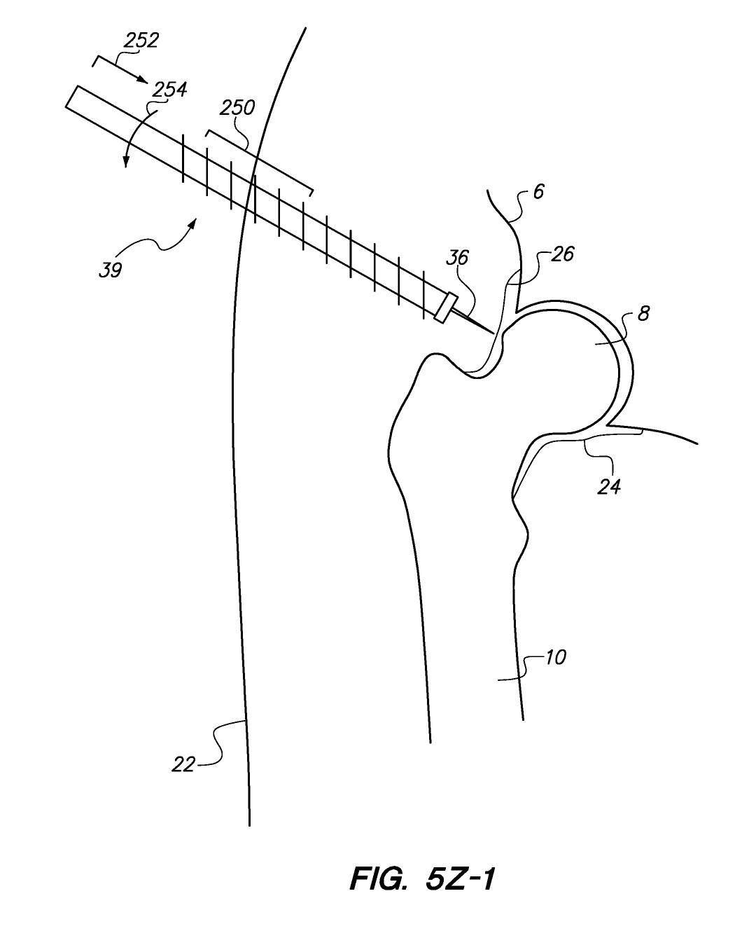

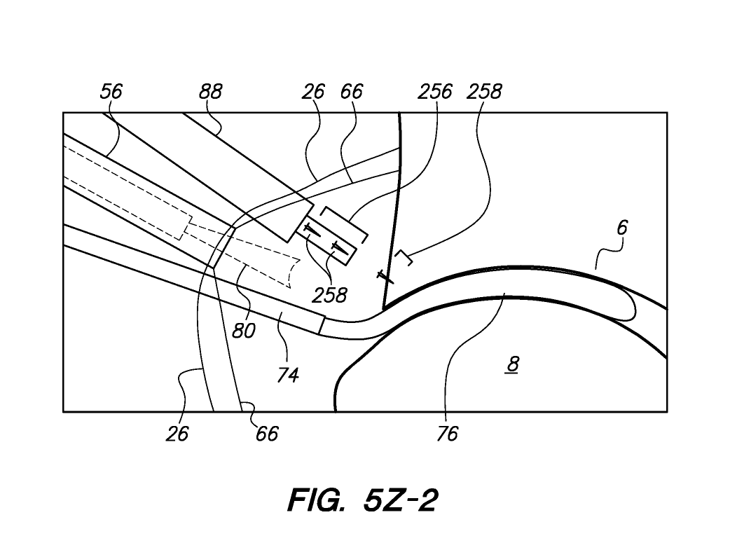

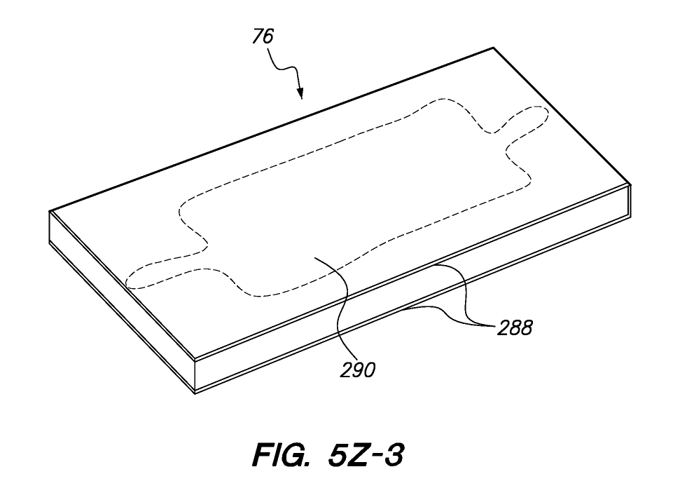

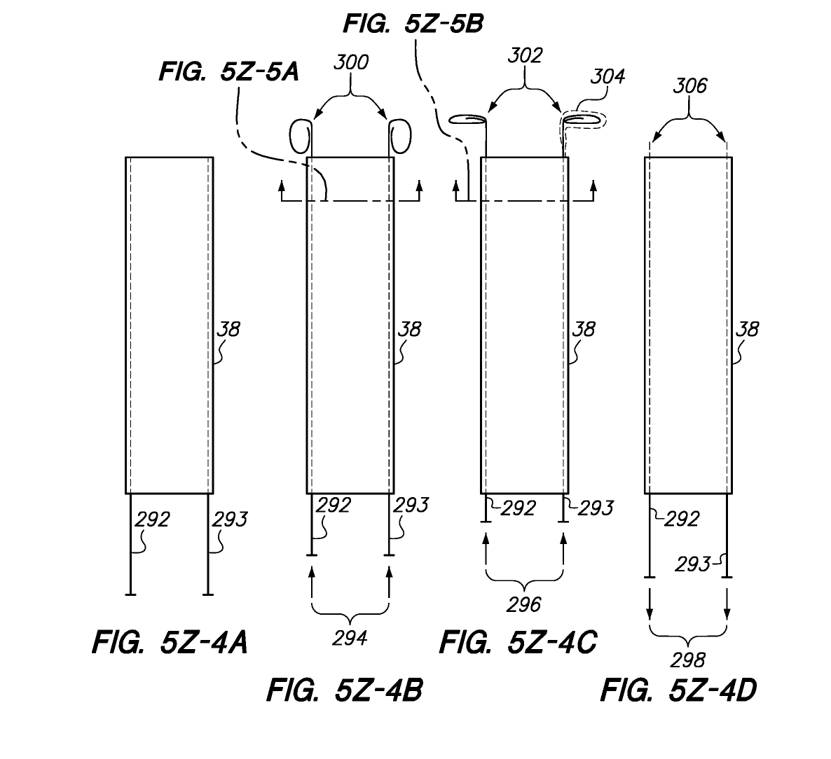

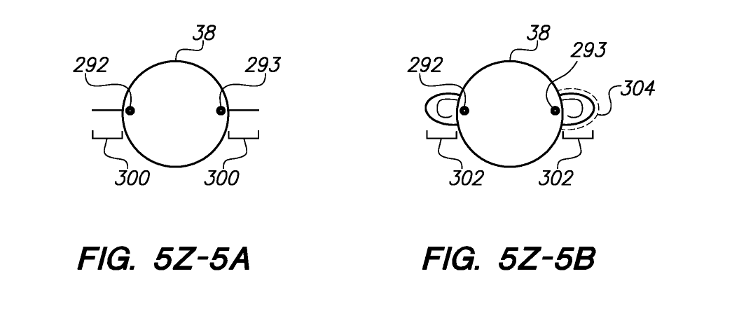

Referring to FIG. 2A, at the extremes of joint mobility, the capsule (24, 26) may become strained, and the geometry of the calcified structures, such as the acetabulum (6) and femur (8), may ultimately cause such structures to become mechanical movement limits. Referring to FIGS. 2B and 2C, certain patients may impinge soft tissue structures as well in such extreme joint positions, such as the labrum (28) around the joint socket formed by the three bones of the acetabulum (6) when loaded against a portion of the neck of the femur (8). Removal of portions (30) of the femoral neck calcified tissue may assist in reducing this type of intersection, as described, for example, in U.S. Patent Application Ser. No. 61/305,519, which is incorporated by reference herein in its entirety. Notwithstanding such procedures, it is desirable in many interventional scenarios to address damaged labral tissue problems, and also address bone geometry configurations which can be optimized with minimally invasive tools. Conventionally, surgery of the hip joint capsule, femoral neck, and labrum is conducted using open surgical techniques which carry with them significant tissue damage and patient recovery downsides. A minority of hip intervention cases are conducted arthroscopically, but as described above, the tools are suboptimal, and this kind of intervention generally is left to specialists in the particular field. Configurations and techniques are described herein for improving the accessibility of challenging arthroscopic diagnostic and interventional procedures, such as hip arthroscopy. Referring to FIG. 3A, after preoperative imaging and patient preparation, an elongate guiding member such as a needle (32) may be advanced (34) across the skin (22) toward the hip joint through one of the known conventional portals (anterior paratrochanteric portal, anterior portal, anterolateral portal, or posterolateral portal, for example); for ease of illustration purposes, a generally lateral portal is depicted. The needle (32) may comprise a conventional radioopaque needle under fluoscopic and/or transcutaneous ultrasound guidance, or may be instrumented with on-board ultrasound sensors, one or more optical coherence tomography (“OCT”) imaging fibers, localization sensors, and direct visualization tools, as described, for example, in U.S. patent application Ser. Nos. 61/311,117 and 61/315,795, each of which is incorporated by reference herein in its entirety. In the embodiment depicted in FIG. 3B, the distal tip (36) of the needle (32) preferably is advanced to a position immediately adjacent, but not past or through, the lateral joint capsule (26). Referring to FIG. 3C, the needle may be utilized in an “over the wire” or “rapid exchange” form to guide a first retraction trocar (38) toward the lateral capsule (26). Suitable elongate trocar members (38) may be substantially rigid to be able to apply orthopaedic surgery scale loads, or may be configured to have a structural modulus configured to maintain a shape substantially similar to an unloaded configuration of the trocar member (38) when navigated through soft tissues, such as muscular tissue, but to deform in sympathy to loads imparted by skeletal calcified tissues (such as a femur, for example) with which it comes into contact. The depicted retraction trocar (38) has a retraction member (40) coupled to its distal portion and configured to controllably expand and retract nearby tissue structures, as described below. The retraction member (40) may comprise an expandable member, such as a balloon or expandable cuff. Such a balloon may be substantially elliptical, spherical, donut-shaped, or frustoconically shaped, and may have a substantially rounded outer shape. An expandable cuff may be flexible or substantially rigid, and may be frustoconically shaped. It also may be expandable, such as by an expanding member such as a balloon, or self expanding (i.e., biased to self expand to an expanded configuration when not restrained in a collapsed configuration). The trocar member (38) may comprise an inflation lumen which may be fluidly coupled to an associated inflatable balloon type retraction member (40). In the depicted embodiment, the retraction member comprises an expandable frustoconical balloon configured to expand into a somewhat hemispherical shape about the distal end of the retraction trocar (38), to create a working volume which may be substantially empty when the frustoconical balloon is expanded, and may be visualized directly and utilized for tool operation and tissue selection and manipulation, as described below. Suitable expandable members configured for minimally invasive delivery and expansion to create a working space endocorporeally are described, for example, in U.S. Pat. No. 5,309,896, which is incorporated by reference herein in its entirety. They may be structurally reinforced with baffles, ribs, and/or metallic ribs or meshwork (for example, braided sheet nitinol embedded into the large surfaces is desirable in certain embodiments to assist with pushability and also retraction loading). Expandable members, and other members for that matter, may be coated with lubricious coatings to enhance slidability relative to other tissues. In other embodiments, expandable members may be specifically textured, or may contain surface texturing elements configured to provide traction relative to tissue structures or other instrumentation. Referring to FIG. 3D, with the needle (32) at least partially withdrawn and the distal portion of the retraction trocar (38) against the lateral capsule (26), the retraction member (40) may be controllably expanded to urge (42) nearby muscular and connective tissue structures laterally, away from the capsule (26)—in effect creating a small operating space from what previously was only a potential space, by expandable member retraction. Referring to FIG. 3E, with further expansion of the expandable member (40), a larger surface area of the muscular and connective tissue is contacted by the proximal aspect of the expandable member, and with slight withdrawal or pulling of the instrument (44), the operating space adjacent the capsule (26) is further enlarged. FIG. 3F depicts further enlargement of the operating volume, in this case facilitated by one or more of instrument withdrawal (44) and further inflation of the distally disposed expandable member (40). Additional expansion and/or retraction may be facilitated by placing a balloon-tipped expandable member through a working lumen of the retraction trocar (38) and inflating/expanding the balloon (48), as shown in FIG. 3G. With an adequate working volume created, interventional tools, such as the sharp-tipped (51) elongate cutting tool (50) depicted in FIG. 3H, may be navigated through the working volume under direct visualization of an image capture device which may be disposed adjacent the apex of the expandable member (40), as described below in reference to FIGS. 5O-5S. The sharp-tipped (51) elongate cutting tool (50) may be utilized for a variety of purposes from tissue debridement to simple perforation of the lateral capsule (26) for further tool advancement or the creation of one or more relaxing incisions generally parallel to the axis of the femoral neck, to assist with controllably loosening an arthritic or contracted joint.

Referring to FIG. 3I, a guidewire (52) may be inserted (and rotated/rolled) through the working lumen of the retraction trocar (38) to assist with further advancement of pertinent tools. For example, in FIG. 3I, the guidewire (52) has been advanced slightly across the lateral capsule (26) in a location selected for further tool advancement, and as shown in FIG. 3J, a second retraction trocar (56) is depicted being advanced (54) over the guidewire (52). In another embodiment, a rapid exchange type of guiding configuration, such as those described in U.S. Pat. No. 6,921,411, incorporated herein by reference in its entirety, may be utilized. Referring to FIG. 3K, with the distal tip (58) of the second retraction trocar (56) advanced (54) across the lateral capsule (26), the guidewire (52) may be at least partially withdrawn (60), leaving the second retraction trocar (56) as a conduit from the outside world to inside of the joint capsule. Referring to FIG. 3L, in an embodiment wherein the extracapsular working volume is no longer needed, the expandable retraction member (40) may be deflated, and the first retraction trocar (38) withdrawn (62).

Referring to FIGS. 3M-3O, the distally disposed retraction member (66) of the second retraction trocar (56), similar in structure, in this embodiment, to the retraction member (40) of the first retraction trocar (38), may be controllably expanded in a manner similar to that described above in reference to the first retraction trocar (38) expandable member (40), and such expansion, along with optional proximal withdrawal loading applied (64) to the proximal aspect of the second retraction trocar (56), creates a working volume within the joint capsule. The working volume may be utilized for tissue manipulation, tool navigation, and visualization/imaging (similar to that described below in reference to FIGS. 5O-5S, but on the other side of the capsule tissue structure 26) of the adjacent structures. Referring to FIG. 3P, another guiding member, such as a needle or guidewire (68) is shown being advanced (70) toward and into the working volume by creating a very small perforation in the retraction member (66). Controllable inflation of the retraction member may be utilized to maintain its shape notwithstanding a small perforation, and in one embodiment, saline may be used to infuse the inflatable retraction member of the depicted configuration to prevent excess gas insufflation of the region. Referring to FIG. 3Q, with the guidewire (68) in place, a joint distraction probe (74) with expandable tip (76) may be advanced over the wire or in rapid exchange form, to access the working volume inside of the capsule. Referring to FIG. 3R, the guidewire has been withdrawn, and as shown in FIG. 3S, the joint distraction probe (74) with expandable tip (76) may be advanced (78) to place the expandable tip (76) within the joint space between the acetabulum (6) and femoral head (8). With the expandable tip (76) in an appropriate joint distraction position, the joint may be gently “jacked open” with inflation of the expandable tip (76), or may be positioned into distraction with manual manipulation of the leg and pelvis—and then retained in distraction with inflation of the distal tip (76), as shown in FIG. 3T.

Referring to FIG. 3U, with the joint distracted in a position wherein the labrum may be manipulated, inspected, and debrided, another elongate tool, such as a grasping tool (80) may be advanced (82) through the working lumen of the second retraction trocar (56), to provide an operator with remote grasping manipulation capabilities at the labrum while the joint remains distracted by the expanded tip (76) of the distraction probe (74). Referring to FIG. 3V, an additional guidewire (84) may be inserted as a guiding member for a third trocar (88), as shown in FIG. 3W, through which an additional elongate tool, such as a cutting tool (94) may be advanced to provide for microsurgical type of tissue manipulation inside of the capsule, as depicted in FIG. 3Y. Referring ahead to FIG. 5Z-2, a tacker instrument (256) may be utilized to tack portions of the labrum into place.

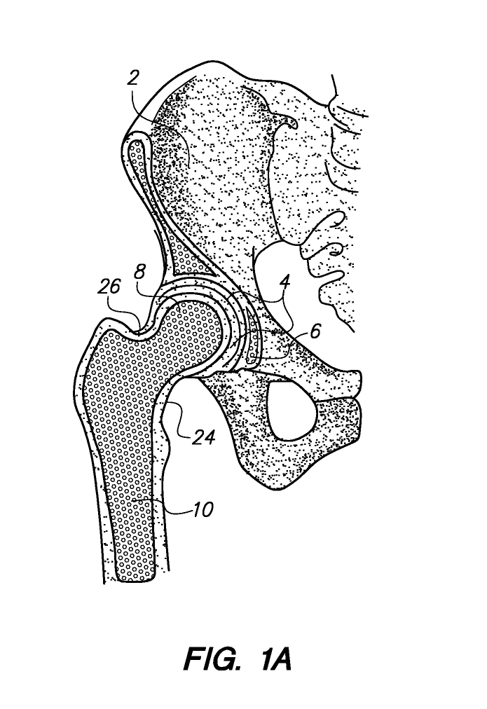

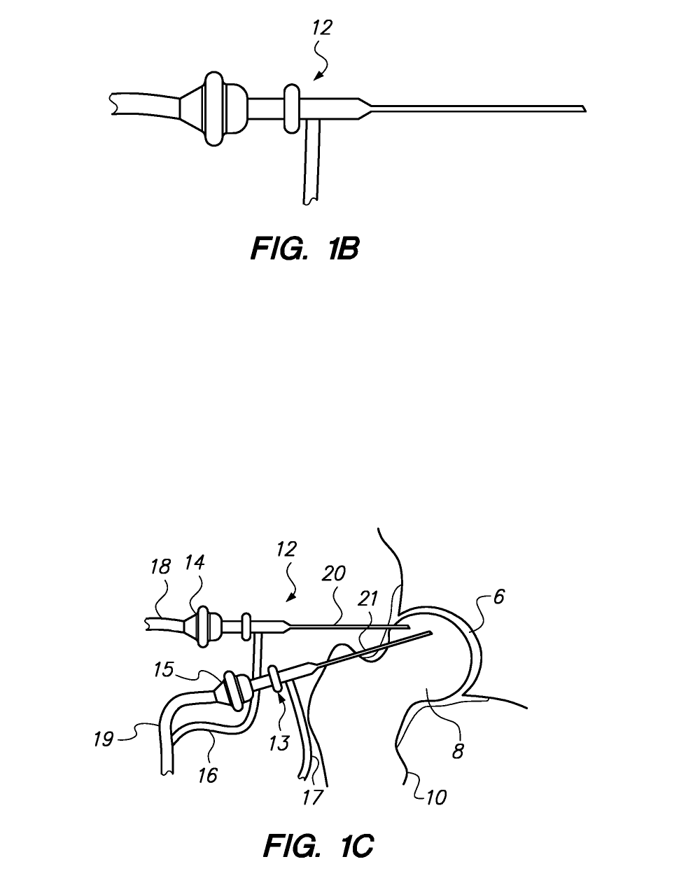

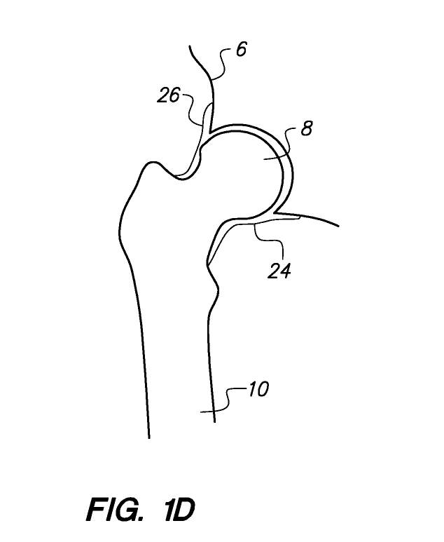

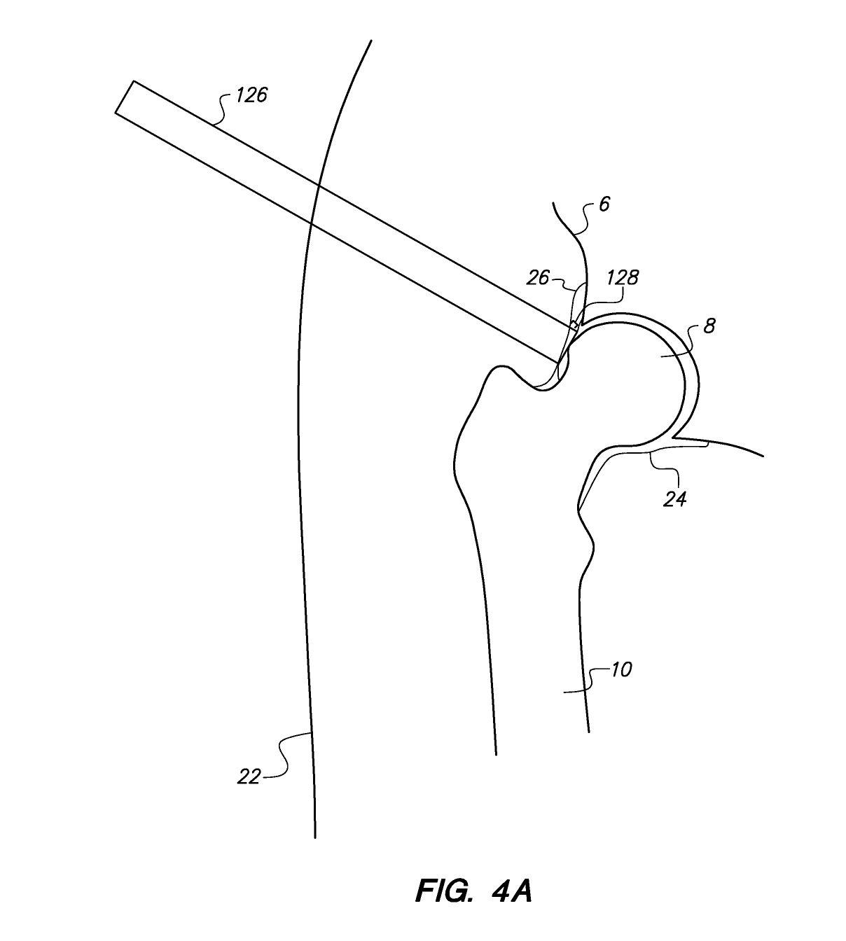

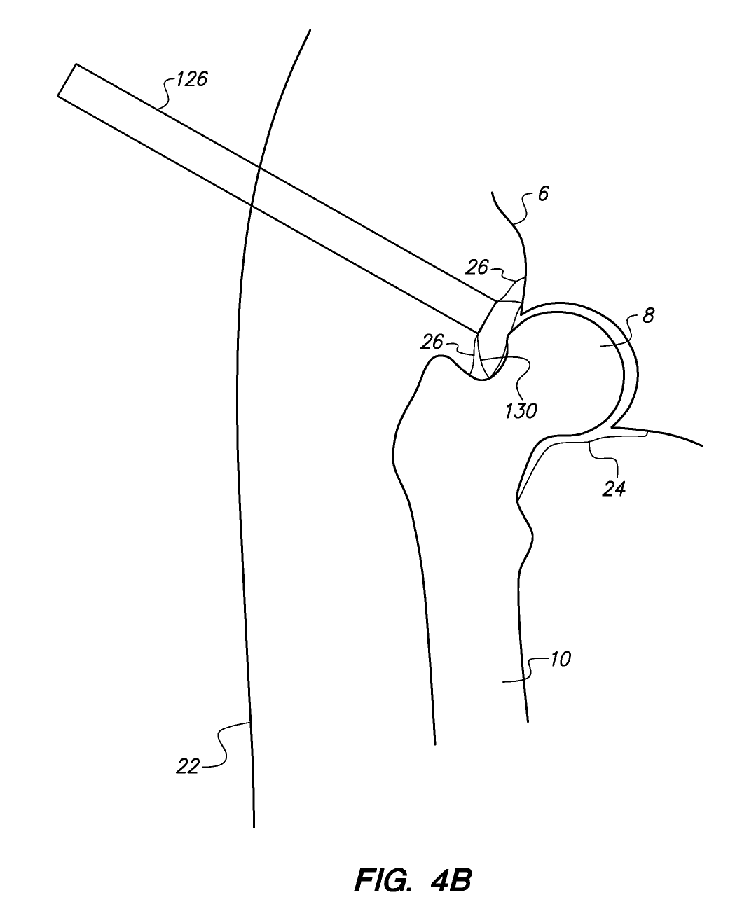

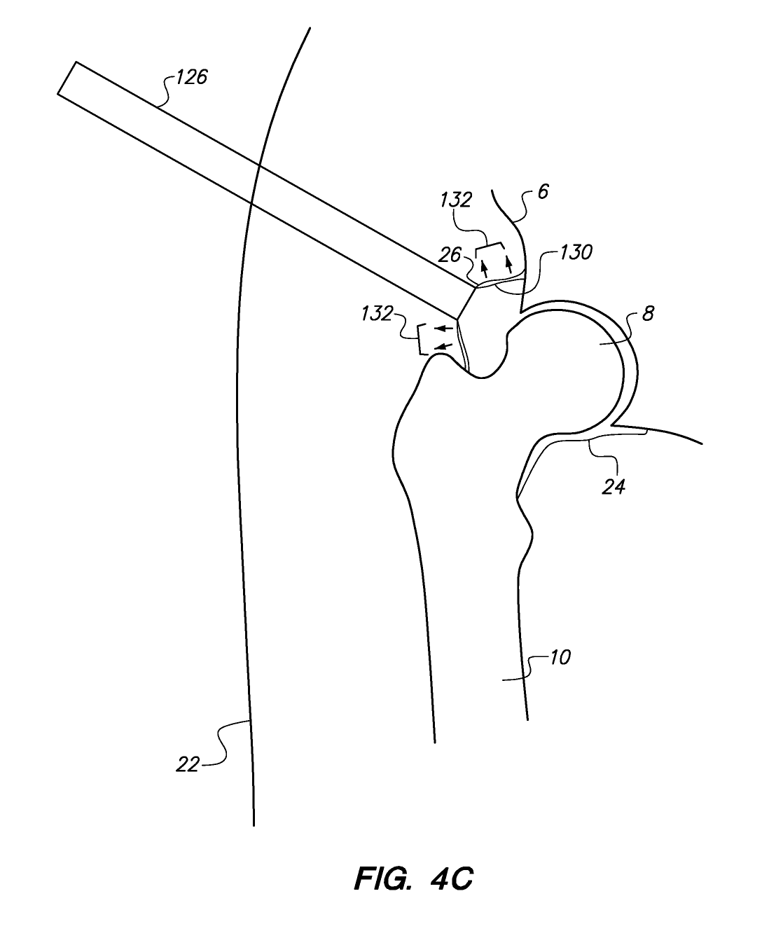

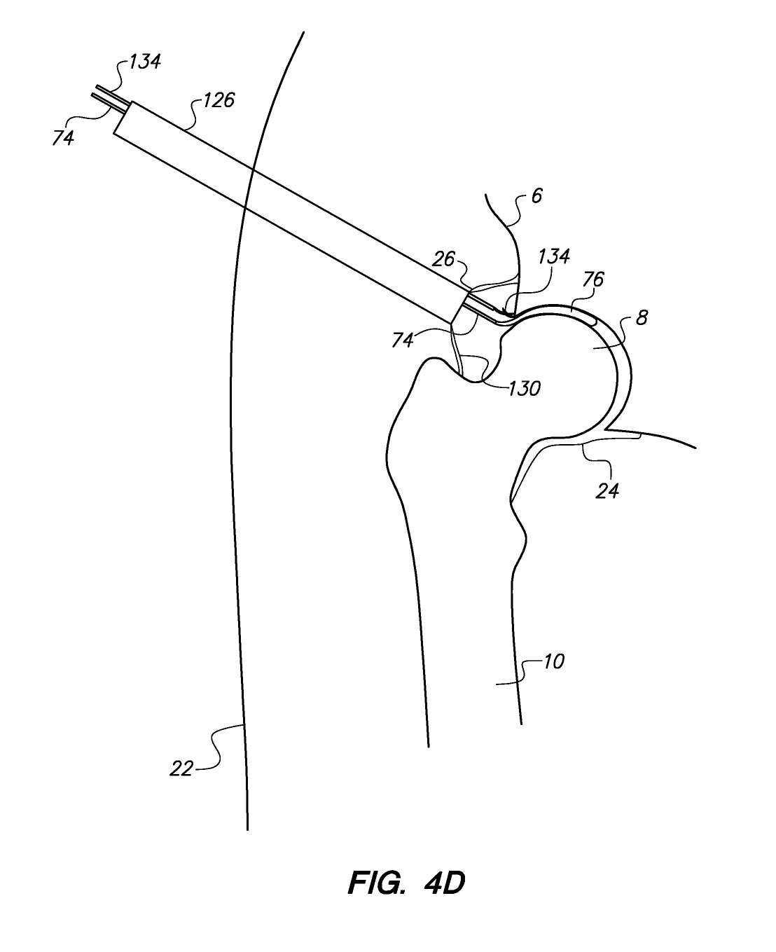

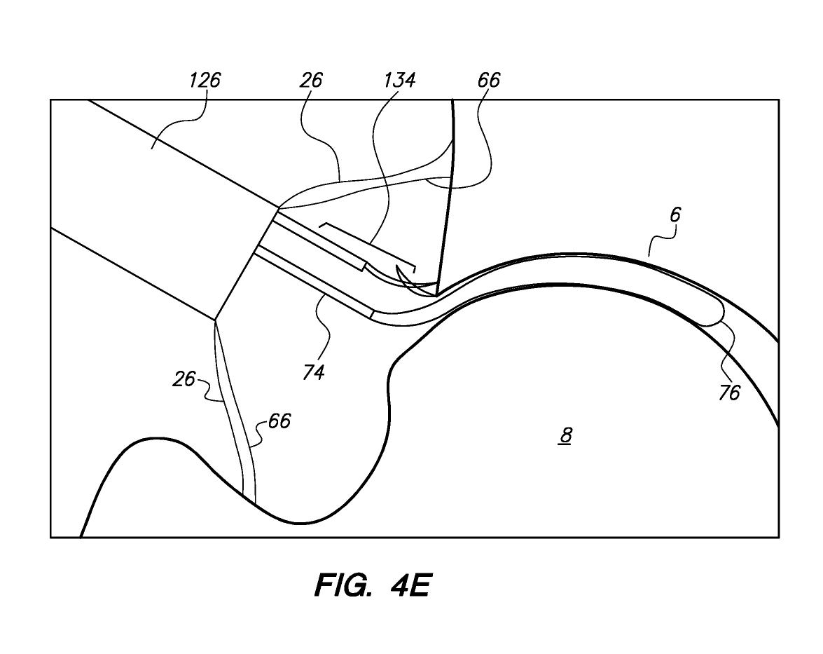

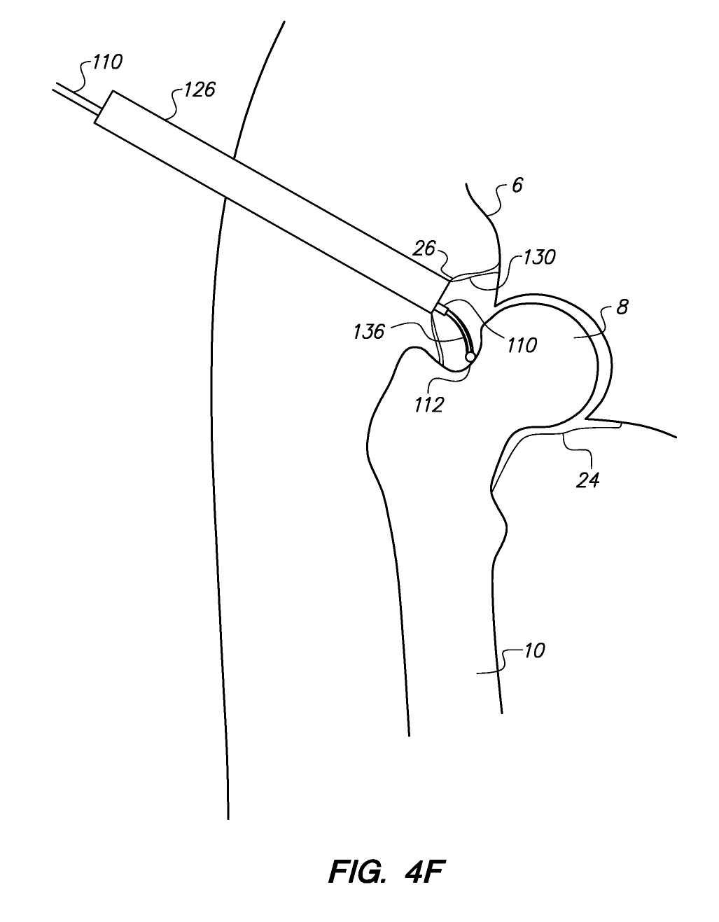

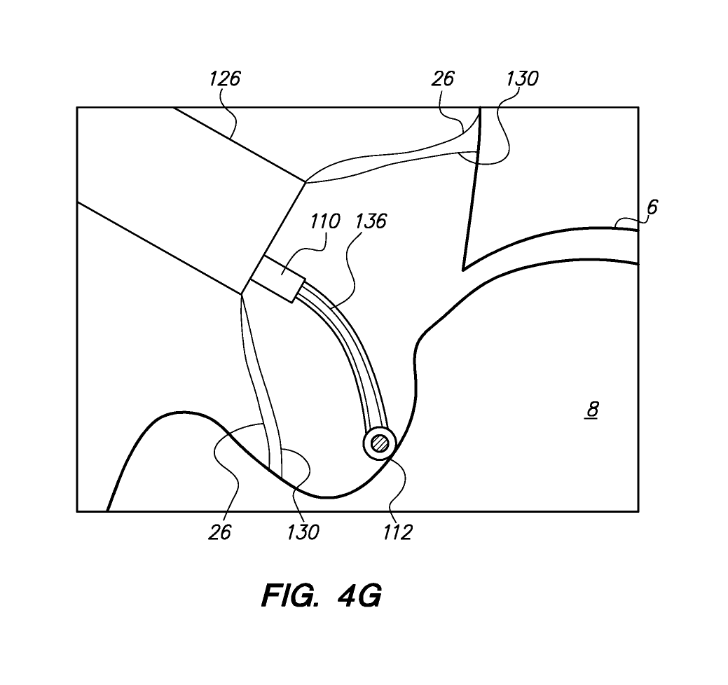

Referring to FIG. 3Z, with the labral intervention completed, the expandable member (76) of the distraction probe (74) may be reduced to its deflated size, after which it may be removed along with the other interventional tools (80, 94), as shown in FIG. 3Z-1. In the event that a reshaping of a portion of the femoral neck is desired, the working volume created by the retraction member (66) of the second retraction trocar (56) may be maintained, and a guiding member such as a needle (102) or guidewire advanced (104) toward the working volume, as shown in FIGS. 3Z-2 and 3Z-3. As described above, insertion of any and all of the elongate tools mentioned herein may be assisted using the imaging and navigation technologies disclosed in the aforementioned incorporated by reference disclosures. Referring to FIG. 3Z-4, with the needle (102) placed across the capsule (26) and into the working volume, a bone cutting tool (108) may be at least partially advanced over the needle (or using the needle as a rapid exchange type of guide) and into the capsule. In the depicted embodiment, the cutting tool (108) comprises a proximal actuating and triggering (114) mechanism, an elongate shaft (110), and a distal burr (112) configured to cut calcified tissue. Other bone sculpting tools and accessories, such as those described in the aforementioned and incorporated 61/305,519, may be utilized to conduct the bone intervention and thereby facilitate a larger non-impinging range of joint motion. Referring to FIG. 3Z-5, after joint mobilization and other inspection or intervention, the cutting tool assembly (112, 110, 108) may be removed (118). Referring to FIGS. 3Z-6 and 3Z-7, the distal retraction member (66) may be deflated as the second retraction trocar (56) is advanced (122) to at least partially unload the nearby tissues and allow for closure of the working volume. Referring to FIG. 3Z-8, with the labral and femoral neck interventions complete, the second retraction trocar (56) is withdrawn (124), leaving the joint ready for rehabilitation, as shown in FIG. 3Z-9.

Referring to FIGS. 4A-4G, another embodiment is depicted wherein similar steps may be utilized to create a working volume directly inside of the capsule (i.e., without first creating an extracapsular working volume as in the embodiment of FIGS. 3A through 3Z-9). The embodiment of FIGS. 4A-4G further differs from that of FIGS. 3A through 3Z-9 in that a single port access configuration is utilized to reduce trauma on the nearby tissue structures and simplify tool exchanges. Referring to FIG. 4A, a retraction trocar (126) with distal retraction member (128) has been positioned using techniques similar to those disclosed above, with a guiding member such as a needle (not shown) being positioned directly across the lateral aspect of the joint capsule (26), followed by an over-the-guiding-member or rapid exchange type of trocar (126) introduction leaving the retraction portion (130) positioned within the joint capsule (26) where it may be controllably expanded, as shown in FIG. 4C, to retract (132) the capsule (26) and create a working volume for further diagnostics and intervention. Referring to FIG. 4D, and the close up view of FIG. 4E, two discrete tools have been advanced through two different working lumens of the retraction trocar (126), to facilitate joint distraction and tissue manipulation at the labrum, using a distraction probe (74) similar to that described above, along with a scissor probe (134) configured for remotely (proximally) actuated scissor cutting action. With the expandable member (76) holding the joint in distraction, the scissor probe (134) may be utilized to debride a damaged labrum—all through one port provided by the proximal confines of the retraction trocar (126). Suitable small-sized scissor probe and grasping type hardware is available from suppliers such as Microfabrica of Santa Clara, Calif. Referring to FIG. 4F, a third working lumen of the retraction trocar (126) may be utilized to facilitate passage of a bone cutting tool similar to that described above, with the exception that the embodiment depicted in FIG. 4F, and close up in FIG. 4G, has a steerable elongate element (136) coupling the burr (112) to the elongate shaft (110) of the bone cutting tool. The steerable elongate element, steerable by virtue of pullwires or the like, may be utilized along with direct visualization provided adjacent the apex of the expandable retraction member (130), similar to the manner in which visualization is described in reference to FIGS. 5O-5S.

Referring to FIG. 5A, a transcutaneous ultrasound transducer (138) with a field of view (140) oriented toward the tissue structure and interventional tools of interest may be utilized preoperatively or intraoperatively to visualize, navigate, and guide the tools relative to pertinent anatomy. Similarly fluoroscopy may be so utilized.

Referring to FIGS. 5B and 5C, as described briefly above, an instrumented needle (142) or other elongate guiding member also may be utilized to navigate pertinent anatomy and instrumentation. Referring to FIG. 5C, one embodiment of an instrumented needle (142) is illustrated wherein a distally-disposed ultrasound transducer (146) may be utilized with a generally forward-oriented field of view (160) to image nearby structures, particularly with roll/rotation of the instrumented needle body to capture a three-dimensional volume within the field of view over a relatively short period of time. An optical fiber (150) may be utilized for OCT imaging (i.e., to determine where nearby tissue structures are located relative to the needle 142 tip, etc.) and/or fiber-Bragg type deflection and/or load detection monitoring, which may be utilized to guide the needle (142) trajectory in a known coordinate system. The depicted embodiment also features a nerve detection sensor electrode, such as those available from Checkpoint Surgical, Inc. under the tradename “Checkpoint Simulator/Locator”. The electrode may also be utilized for impedance-based tissue contact monitoring. The depicted embodiment also features a localization sensor, such as those available from St. Jude Medical (potential difference based system under the tradename “EnSite” ®), or those available from the Biosense division of Johnson and Johnson, Inc. (electromagnetic flux based system under the tradename “CartoXP” ®). Such localization sensor may be utilized in a known coordinate system to assist with relative positioning of the needle (142) and other instruments and tissue structures. The OCT fiber (150) is proximally coupled to an OCT interferometry system (194), and may be signal split to also be coupled with a fiber Bragg monitoring system (i.e, with time multiplexing to operate both modalities, etc). The ultrasound transducer is operatively coupled via an electrical lead (158) to an ultrasound system (186), such as those available from the Acuson division of Siemens Medical Systems GmbH. The localization sensor (154) may be operatively coupled to a localization system using an electrical lead (156). The nerve sensor electrode (148) may be operatively coupled to a nerve detection system (190) also using an electrical lead. Each of these subsystems (186, 190, 194, 192) may be operatively coupled with various electrical leads to the control/display interface module (188) of the system.

Referring to FIG. 5D, a retraction trocar (38, 56) with expandable distal tip (40, 66) is depicted with a needle (32, 142) positioned through its working lumen. Referring to FIG. 5E, a similar retraction trocar (38, 56) with expandable distal tip (40, 66) is depicted with a guidewire (52, 68, 84, 166) passed through an obturator (164), which is passed through the working lumen of the retraction trocar (38, 56). In practice, embodiments such as these depicted in FIGS. 5D and 5E may be inserted through muscular and connective tissue to access joints such as the hip, as shown, for example, in FIGS. 3 and 4. Referring to FIGS. 5F, and 5G, however, it may be desirable to have an expandable member (168), such as a controllably-expandable cylindrical balloon structure, coupled to the retraction trocar (38, 56), to enable the operator to further dilate and press away tissues that may surround the retraction trocar (38, 56). Such expansion action may be utilized after full insertion of the retraction trocar (38, 56), or may be used interactively during insertion to improve insertability of the retraction trocar (38, 56). FIGS. 5H and 51 depict cross sectional views to show the expansion and potential dilation in one embodiment. Such dilation may also improve the interactive response of elongate instruments passed through the subject tissue structures, such as the retraction trocar (38, 56) itself. Referring to FIG. 5J, a multi-section expandable member (170) is shown having three independently controllably inflatable portions to allow for asymmetric dilation action, as well as inchworm type insertion activity with serial expansion and retraction of the inflatable portions. Many shapes may be desirable for the expandable member, such as the bi-lobed variation (172) shown in FIG. 5K, which would facilitate remote center of motion activity of the retraction trocar (38, 56) with the remote center near the center of the retraction trocar (38, 56); in other words, the nearby tissues would be more dilated near the peripheral aspects of the retraction trocar (38, 56), which would have the most motion in a remote center of motion configuration with the remote center at approximately the middle of the retraction trocar (38, 56). Any of the expandable member balloon structures herein may comprise surface treatments to facilitate easy sliding (i.e., lubricious coatings or materials such as PTFE or Teflon), traction (i.e., having surface texturing, embedded ribs, wires, or mesh, and the like), or combinations thereof in nonhomogeneous/directional applications. Expandable members such as any of those featured in FIG. 5G (168), 5J (170), or 5K (172) may also comprise one or more cutting surfaces or elements, such as prominent mechanical cutting features (prominent when the expandable members are in an expanded configuration) configured to facilitate side-directed dissection or laceration of nearby soft tissues (such as capsular connective tissue) upon inflation of the associated expandable members, as described, for example, in various cutting balloon references for other medical applications, such as U.S. Pat. Nos. 5,797,935, 5,196,024, 5,112,305, 5,616,149, 7,686,824, 7,691,119, 7,070,576, 7,291,158, 7,632,288, 7,754,047, and 7,758,604, each of which is incorporated by reference herein in its entirety.

Referring to FIG. 5L, and in cross section FIG. 5M, an expandable member may also be configured to have one or more ultrasound transducers operatively coupled thereto. The embodiment depicted in FIGS. 5L and 5M has two sets (180, 182) of four ultrasound transducers coupled to the flexible balloon substrate. A sample field of view (184) in both orthogonal directions is depicted in FIGS. 5L and 5M. Circuitry and hardware pertinent to the ultrasound transducers may be coupled to the balloon substrate, or may be directly embedded into the substrate using conformal electronic processing such as that available from MC10, Inc. of Cambridge, Mass., and may be proximally coupled to an ultrasound system (186) and ultimately a control/display interface (188) using electronic leads (176, 178).

Referring to FIGS. 5N-5O, a retraction trocar (38) is illustrated in a similar scenario to that shown in FIG. 3D-3F with the exception that trocar instrumentation features re highlighted further in the closer-up views of FIGS. 5N and 5O, wherein the retraction member (40) is utilized to retract promimally, away from the depicted joint capsule layer (26), a layer of muscle tissue (196). Referring to FIG. 5O, an optical image capture device, such as a CCD, CMOS, or other imaging chip, or an optical imaging fiber bundle, is located at the apex of the retraction member (40) to provide a forward-oriented field of view to directly visualize activities within the working volume of the expandable retraction member (40). FIGS. 5P-5R illustrate further magnified views. As shown in FIG. 5P, the image capture device (200) has a field of view (198) configured to capture substantially all of the working volume created by the expanded form of the retraction member (40). The image capture device (200) may be configured to have a field of view (198) extending away from the distal end of the elongate trocar member (38) and at least partially in alignment with a longitudinal axis of the trocar member (38). The image capture device may comprise an ultrasound transducer, an optical imaging device, such as an image capture chip or optical imaging fiber bundle, or an OCT imaging fiber. As described below, an OCT fiber may be additionally or alternatively positioned adjacently. A fluid reservoir containing a transparent fluid such as saline may be fluidly coupled to the working volume and configured to controllably flush the working volume with such transparent fluid, thereby clearing the working volume of other fluids which may be nontransparent from an imaging perspective. The working lumen (208) is also depicted, defined through the elongate retraction trocar (38) body. FIG. 5Q features a cross sectional view showing that the retraction trocar may be instrumented with an image capture device (200), illumination source (i.e., a light 206), an OCT imaging fiber (150), an irrigation head (202) for distributing saline and/or liquid medicines or treatments (including but not limited to stem cells, antiinflammatories, chondrocytes, bone growth agents, etc), and an aspiration/vacuum port (204) for evacuating fluids, gases, or other substances from the working volume. Referring to FIG. 5R, the retraction member of the retraction trocar may comprise a frustoconical balloon which may or may not have a top or lid (212), depending upon the procedural value of having unhindered forward access past the working volume contained within the frustoconical shape. One or more inflation lumens (210) may be utilized to control the pressure within the frustoconical balloon member, which may have one or more bladders.

Referring to FIG. 5S, an embodiment is depicted wherein several other tools and/or features may be utilized to create and maintain a working volume. In the depicted embodiment, a proximal collar member (220) may be slidably coupled around the proximal shaft of the retraction trocar (38) to place the tissue between the skin surface (22) and the expandable retraction member (40) into compression (218, 42)—and the retraction trocar may be pulled proximally (222) to assist with the opening/creation of the working volume. Further, an elongate pusher member (216) with atraumatic distal tip portion (214) may be pushed toward the hip joint and utilized to further “jack open” and create a working volume.

Referring to FIGS. 5T-5X, 5Z-1, and 5Z-4A through 5Z-5B, notwithstanding the illustrations and discussion herein about a frustoconical balloon-like expandable retraction member (40, 66), other retraction members or means may be utilized to accomplish similar results, and are within the scope of this invention. For example, referring to FIG. 5T, an embodiment similar to that of FIG. 5S is depicted, with the exception that the expandable retraction member (226) comprises a substantially spherical balloon. Many other balloon shapes and materials types are suitable, including substantially elastic balloons, substantially inelastic balloons, combination balloons, and nonhomogeneous balloons. Referring to FIG. 5U, a mechanical retraction means is provided wherein one or more retraction members (230) may be rotated into a deployed position somewhat akin to the action of a hardware toggle bolt, to assist with mechanical retraction and creation of a working volume from what previously was only a potential space. A close up cross sectional view is shown in FIG. 5V, including one or more image capture devices (200; one is shown in the embodiments of FIGS. 5V and 5Q, but in other embodiments, stereoscopic cameras may be utilized to increase imaging redundancy and also provide some depth perception), one or more light ports (206), an OCT fiber (150), an irrigation port (202), and an aspiration/vacuum port (204). The deployed retraction members are also depicted in the cross sectional view of FIG. 5V. FIGS. 5W and 5X illustrate one embodiment of a deployment paradigm for the retraction members (230), wherein in an insertion configuration, as illustrated in FIG. 5W, a set of pullwires (232) is left untensioned. When deployment is desired, the pullwires may be tensioned (236) as shown in FIG. 5X, and due to their coupling with the retraction members by virtue of the body of the retraction trocar (38), such tension causes the members (230) to rotate (228) into position for retraction. Retrieval only requires release of the tension (236) and proximal pullout of the instrument.

Referring to FIG. 5Y, in one embodiment the expandable distal portion (238) of the distraction probe (74) may comprise a plurality of individually controllably inflatable cells or small bladders (240)—somewhat akin to controllable “bubble wrap”. Referring to FIG. 5Z, the cells (240) may be individually inflatable, or inflatable in groups; the configuration of FIG. 5Z has three separate inflation lumens (242, 244, 246) providing pressure and flow to three groups of the cells which may be controllably inflated to provide for a customized retraction using an inflation control system (248) which may be operatively coupled to an operator computer control workstation (not shown).

Referring to FIG. 5Z-1, a threaded (250) and lubriciously coated outer surface of a retraction trocar (39) may provide not only for relatively easy trocar insertion (i.e., by twisting 254 and some relatively low insertion loading 252), but also for relatively good interfacial purchase against the tissue, allowing for proximal tensioning of the trocar (39) to assist with retraction of the associated tissue and creation of a working volume. A somewhat similar threaded outer shape configuration is featured on the orthopaedic fixation product sold under the tradename “Suretac” (®) by Smith and Nephew, Inc.

Referring to FIG. 5Z-2, a small tacking device, such as that available from Covidien, Inc., under the tradename “Tacker” (®) may be utilized to deploy small tacks (258), preferably comprising a bioinert material such as titanium or a bioresorbable polymer such as poly lactic acid, poly glycolic acid, or polylactic glycolic acid.

Referring to FIG. 5Z-3, to improve the “pushability” of inflatable distraction and retraction members, one or more bladders (290) defined within one or more layers of polymeric or other material (such as Dacron) may be reinforced with small metallic rib or wire structures (288), as shown in FIG. 5Z-3 about each of the edges of the depicted expandable structure (76).

Referring to FIGS. 5Z-4A through 5Z-5B, an additional retraction member configuration is shown wherein one or more pre-treated nitinol wires may be controllably inserted through a lumen of a retraction trocar member (38) and configured to initially form loops in an orientation (300) that is substantially parallel to the longitudinal axis of the trocar member (38), but which change orientation again to an orthogonal orientation (302), with further insertion of the wires. Referring to FIG. 5Z-4A, with the wires (292, 293) pulled proximally and not protruding distally from the trocar body (38), there is no retraction distally. With a first incremental push (294) of the wires, as in FIG. 5Z-4B, the parallel loops are formed (also shown in cross sectional view in FIG. 5Z-5A). With a second incremental push (296), the loops are orthogonally reoriented (302), as shown in FIG. 5Z-4C, and in cross section in FIG. 5Z-5B. One or more “socks” or liners (304) may be provided to ensure that when in the fully deployed (i.e., oriented to function as a retraction paddle similar to the retraction members 230 depicted in FIG. 5V), they do not damage nearby tissue structures (i.e., particularly with the distal tip). As shown in FIG. 5Z-4D, with retraction (298), the wires return to a non-retracting form. Thus yet another retraction trocar embodiment is presented in FIGS. 5Z-4A through 5Z-5B.

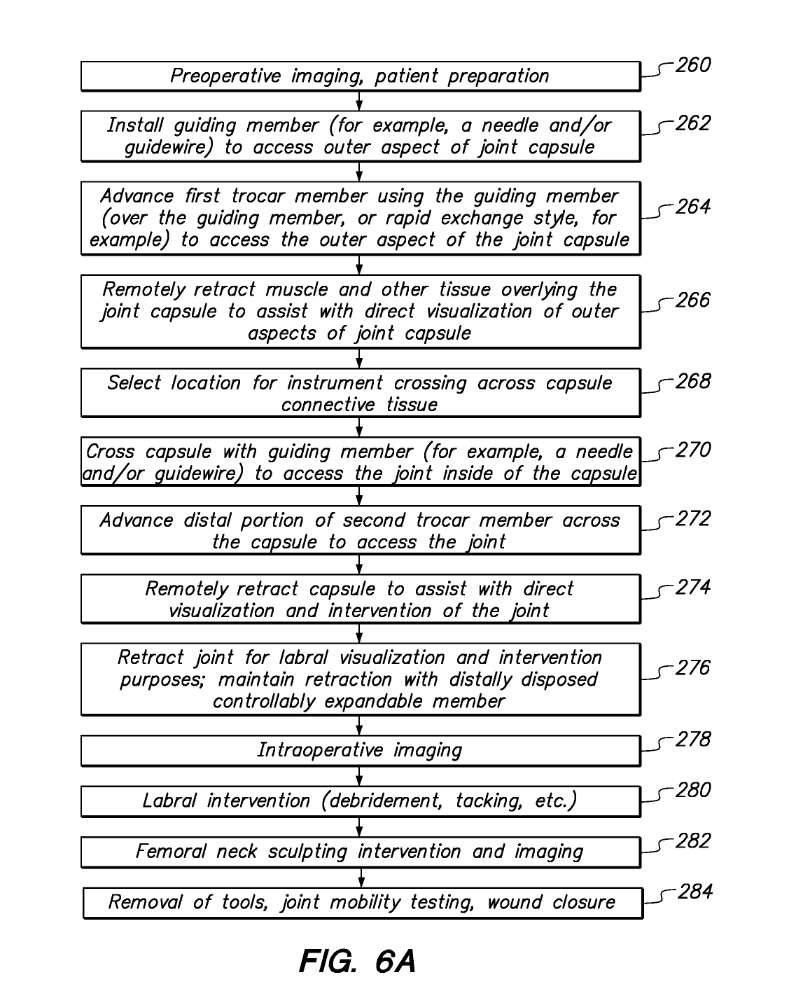

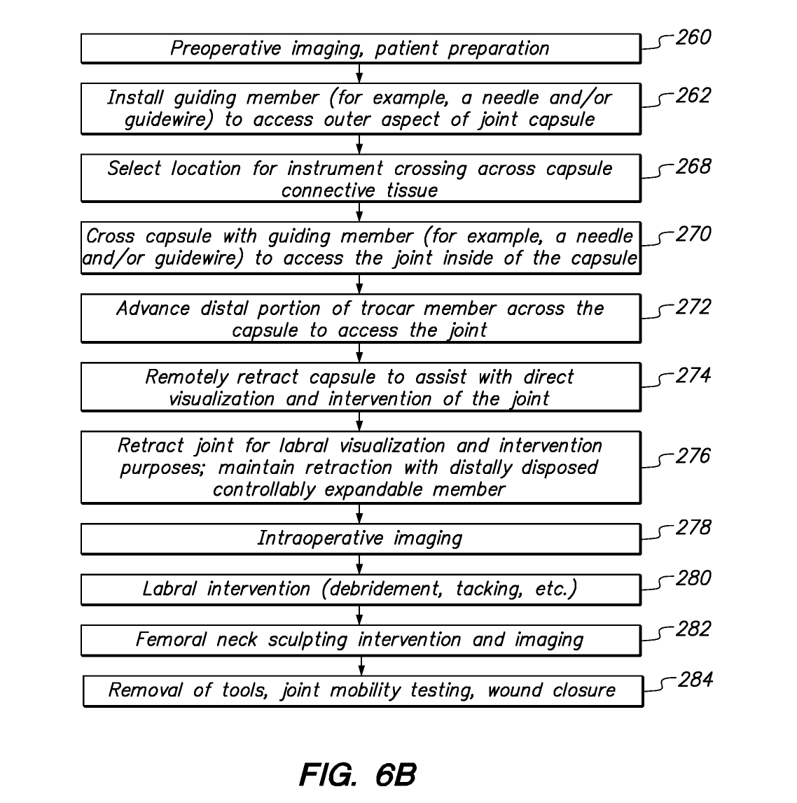

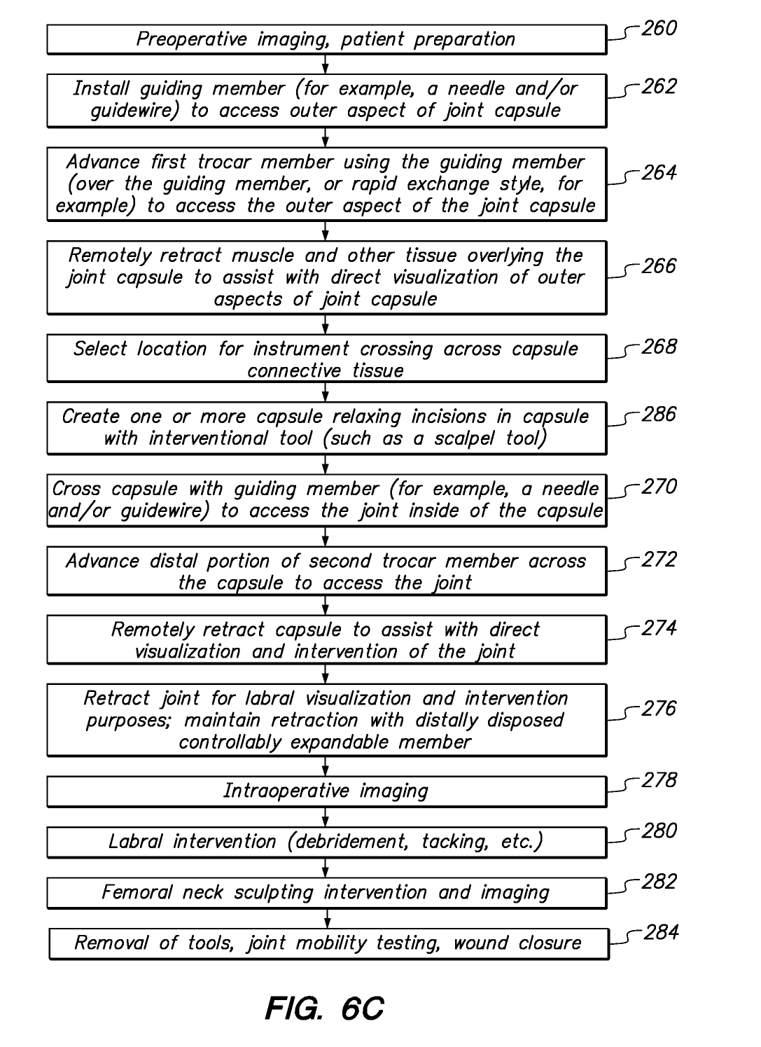

Referring to FIG. 6A, a method for conducting an arthroscopy procedure incorporating various of the above configurations and/or steps is illustrated. After preoperative imaging (for example, CT, MRI, transcutaneous ultrasound, Xray, fluoroscopy) and patient preparation (for example, placement in an operating room for a lateral or supine approach for hip arthroscopy) (260), a guiding member, such as a needle, guidewire, trocar, or the like may be advanced to access the outer (lateral) aspect of the joint capsule (this is in a procedure embodiment wherein the physician desires to visualize and potentially intervene at certain outer aspects of the joint capsule before proceeding past the threshold of the capsule and into the joint) (262). With the guiding member in place, a first retracting trocar member may be advanced into place (264), using an over-the-wire type of technique, rapid exchange type of technique, or in another embodiment, without a guiding member (through the use of an image guided trocar having instrumentation similar to that described in the aforementioned incorporated by reference patent applications 61/311,117 and 61/315,795). A portion of the trocar (for example, an expandable member, a repositionable mechanical element, and/or threaded outer surface) may then be utilized to retract proximally muscle and other tissues overlying the capsule to assist with direct visualization (using, for example, an image capture device) of outer aspects of the joint capsule (266). With such visualization (and also, in one embodiment, with the assistance of other imaging modalities such as ultrasound, OCT, fluoroscopy, and the like), a location for crossing the capsule may be selected (268), and the capsule may be crossed with an elongate guiding member (270) such as a guidewire or needle to access the joint. With the joint accessed, a second trocar member may be advanced toward the endocorporeal operating theater over the wire or via rapid exchange techniques (272) to provide trocar access to the joint. A portion of the trocar (for example, an expandable member, a repositionable mechanical element, and/or threaded outer surface) may then be utilized to retract proximally capsular and other tissues overlying the joint to assist with direct visualization (using, for example, an image capture device) and intervention of aspects of the joint (274). Interventional tools may be brought into the working volume for tissue manipulation purposes (i.e., to address a labrum debridement or repair challenge), such as grasper, scissor, and tacker tools, along with remotely controllable joint distraction tools, which may be utilized to open, or hold open, the subject joint (for example, during labral intervention at the hip) (276) during intraoperative imaging (278) and interventional (280) steps. Subsequently, the distraction may be released and additional tools may be brought into the operating theater to sculpt calcified tissue, such as the neck of the femur to reduce impingement (282), after which the tools may be removed, joint mobility tested, and wound closure conducted (284).

FIG. 6B depicts a similar intervention, wherein the steps (264, 266) of visualizing the outer aspects of the joint capsule may be skipped, to provide direct access to the inside of the joint. FIG. 6C depicts a similar intervention as described in reference to FIG. 6A, with the exception that one or more relaxing incisions may be created (286) using the working volume created with the first retracting trocar at the outer aspect of the capsule.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, hydrophilic gel or silicones) may be used in connection with various portions of the devices, such as relatively large interfacial surfaces of movably coupled parts (i.e., such as the surfaces between the above described arthroscopic instruments and tools which may be placed through working channels or lumens 28 defined by the arthroscopic instruments, as described in reference to FIGS. 3B, 3D, 4B, 5B, 6B, 6D, 7C, 8B, 8D, 9B), if desired, for example, to facilitate low friction manipulation or advancement of such objects relative to other portions of the instrumentation or nearby tissue structures. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and the include plural referents unless the specifically stated otherwise. In other words, use of the articles allow for at least one of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

1. An apparatus for conducting an orthopaedic intervention, comprising:
a. an elongate trocar member having proximal and distal ends, a longitudinal axis, and defining a first working lumen therethrough; and
b. a controllably expandable distal member coupled to the distal end of the elongate trocar member and configured to be expanded from a collapsed state, wherein the expandable distal member has an outer diameter similar to that of the trocar member, to an expanded state, wherein the expandable distal member has an outer diameter larger than that of the trocar member.
